(19) United States
(12) Patent Application Publication
(10) Pub. No.: US 2024/0269172 A1
(43) Pub. Date: Aug. 15, 2024

(54) Methods and Compositions for Treatment of Intervertebral Disc Disease

(71) Applicant: DiscCath, LLC, New York, NY (US)

(72) Inventor: Gregory E. Lutz, Pennington, NJ (US)

(21) Appl. No.: 18/436,797

(22) Filed: Feb. 8, 2024

Related U.S. Application Data
(60) Provisional application No. 63/484,288, filed on Feb. 10, 2023.

(51) Int. Cl. A61K 35/16; A61K 35/15; A61K 35/19; A61K 45/06

(52) U.S. Cl. CPC A61K 35/16; A61K 35/15; A61K 35/19; A61K 45/06

(57) ABSTRACT

Provided herein are, in various embodiments, methods and compositions for treating a tissue disease, e.g., intervertebral disc disease. In certain embodiments, the methods and compositions comprise platelet-rich plasma (PRP). In further embodiments, the PRP is leukocyte-rich PRP, granulocyte-rich PRP, lymphocyte-rich PRP, monocyte-rich PRP, or a combination thereof. As described herein, the quantity of leukocytes and other nucleated cells and platelets exceeds that of commercially available PRP compositions.

### RELATED APPLICATION

This application claims the benefit of U.S. Provisional Application No. 63/484,288, filed on Feb. 10, 2023. The entire teachings of the above application are incorporated herein by reference.

### BACKGROUND

Degenerative disc disease (DDD) is the number one cause of chronic low back pain (CLBP). The point prevalence of activity-limiting lower back pain about 580 million people worldwide and chronic, low back pain is now regarded as the number one cause of disability globally. Drugs and surgery have not been the cure for the majority of patients with CLBP. Despite poor outcomes, the number of lumbar fusions for degenerative conditions has increased 276%. The economic consequences are significant for the individual and for the healthcare system as a whole, costing the economy $560-$630 billion annually.

Healthcare systems have not made meaningful advancements in managing “non-fatal” conditions such as CLBP. The root causes of CLBP need to be reconsidered, and treatments targeted directly at them to truly find a cure. Simple, safe, cost-effective, scalable treatments that are durable in their effect are needed. Healthcare systems need to shift their approach to musculoskeletal diseases away from volume-based palliative treatments to value-based root cause treatments that create sustained improvements. As disclosed herein, intradiscal leukocyte-rich platelet-rich plasma (LR-PRP) has the potential to be a value-based root cause treatment for many patients with symptomatic lumbar disc disease.

### SUMMARY

Described herein are methods and compositions for treating, preventing, or ameliorating a tissue disease. The present disclosure contemplates a method to regenerate tissue that does not rely on circulation of blood for perfusion and is prone to bacterial infection with a blood derived product sourced from the patient. In some aspects, the blood is centrifuged to create a concentrate of leukocytes, other nucleated cells, and platelets. In some aspects, the blood product is delivered locally to the area of tissue damage and/or infection. The methods and compositions disclosed herein may be useful in the treatment of one or more intervertebral discs of the spine of a subject. Administration may occur pre-operatively, peri-operatively, or post-operatively. The treatment may be useful in the treatment of already damaged tissue or in the prevention of tissue damage. The present disclosure also contemplates the co-administration with an antibiotic for additional antimicrobial benefits. In certain aspects, the compositions and methods of the disclosure include leukocyte-rich platelet rich plasma that has an increase in certain blood components above baseline. In some examples, the increase is at least about 5× to at least about 20×.

In one aspect, the present disclosure provides a method of treating, preventing, or ameliorating a tissue disease in a subject, comprising administering to the subject a therapeutically effective amount of a platelet-rich plasma (PRP) composition comprising: plasma; platelets at a concentration of at least or at least about 5× to at least or at least about 75× the concentration of platelets in whole blood; and nucleated cells at a concentration of at least or at least about 5× to at least or at least about 75× the concentration of nucleated cells in whole blood, wherein the nucleated cells comprise monocytes, lymphocytes, granulocytes, or a combination thereof.

In some embodiments, the concentration of platelets is at least or at least about 5×, 10×, 15×, 20×, 25×, 30×, 35×, 40×, 45×, 50×, 55×, 60×, 65×, 70×, or 75× the concentration of the platelets in whole blood. In some embodiments, the concentration of nucleated cells is at least or at least about 5×, 10×, 15×, 20×, 25×, 30×, 35×, 40×, 45×, 50×, 55×, 60×, 65×, 70×, or 75× the concentration of the nucleated cells in whole blood. In some embodiments, the concentration of monocytes is at least or at least about 5×, 10×, 15×, 20×, 25×, 30×, 35×, 40×, 45×, 50×, 55×, 60×, 65×, 70×, or 75× the concentration of the monocytes in whole blood. In some embodiments, the concentration of granulocytes is at least or at least about 5×, 10×, 15×, 20×, 25×, 30×, 35×, 40×, 45×, 50×, 55×, 60×, 65×, 70×, or 75× the concentration of the granulocytes in whole blood.

In some embodiments, the concentration of the platelets, the nucleated cells, the monocytes, the lymphocytes, the granulocytes, or a combination thereof is each at least 10× the concentration in whole blood. In some embodiments, the concentration of platelets is at least or at least about 10× the concentration of the platelets in whole blood. In some embodiments, the concentration of nucleated cells is at least or at least about 10× the concentration of the nucleated cells in whole blood. In some embodiments, the concentration of monocytes is at least or at least about 10× the concentration of the monocytes in whole blood. In some embodiments, the concentration of granulocytes is at least or at least about 10× the concentration of the granulocytes in whole blood. In some embodiments, the concentration of platelets is at least or at least about 10× the concentration of the platelets in whole blood and the concentration of nucleated cells is at least or at least about 10× the concentration of the nucleated cells in whole blood. In some embodiments, the concentration of platelets is at least or at least about 10× the concentration of the platelets in whole blood and the concentration of nucleated cells is at least or at least about 5× the concentration of the nucleated cells in whole blood.

In some embodiments, the treating, preventing, or ameliorating the tissue disease further comprises regenerating tissue.

In some embodiments, the treating, preventing, or ameliorating the tissue disease further comprises reducing bacterial dysbiosis.

In some embodiments, the bacterial dysbiosis comprises an overgrowth of Pseudomonas, Streptococcus, Cutibacterium, or a combination thereof. In some embodiments, the bacterial dysbiosis comprises an overgrowth of Pseudomonas. In some embodiments, the bacterial dysbiosis comprises an overgrowth of Streptococcus. In some embodiments, the bacterial dysbiosis comprises an overgrowth of Cutibacterium. In some embodiments, the bacterial dysbiosis comprises an overgrowth of Pseudomonas, Streptococcus, and Cutibacterium.

In some embodiments, the tissue is avascular tissue. In some embodiments, the tissue is intervertebral disc tissue. In some embodiments, the avascular tissue is an intervertebral disc. In some embodiments, the tissue is more than one intervertebral disc. In some embodiments, the tissue is already damaged. In some embodiments, the tissue may be at risk of becoming damaged.

In some embodiments, the PRP is delivered locally to the area of tissue damage and/or infection. In some embodiments, the PRP may be delivered pre-operatively, peri-operatively, or post-operatively.

In some embodiments, the PRP is delivered intradiscally through an injection.

In some embodiments, the PRP is derived from whole blood. In some embodiments, the PRP is derived from a subject's own whole blood.

In some embodiments, the PRP is administered in combination with at least one other therapeutic. In some embodiments, the at least one other therapeutic is an antibiotic. In some embodiments, the PRP is administered in combination with an antibiotic.

In another aspect, the present disclosure provides a platelet-rich plasma (PRP) composition comprising: plasma; platelets at a concentration of at least or at least about 5× to at least or at least about 75× the concentration of platelets in whole blood; and nucleated cells at a concentration of at least or at least about 5× to at least or at least about 75× the concentration of nucleated cells in whole blood, wherein the nucleated cells comprise monocytes, lymphocytes, granulocytes, or a combination thereof.

In some embodiments, the concentration of platelets is at least or at least about 5×, 10×, 15×, 20×, 25×, 30×, 35×, 40×, 45×, 50×, 55×, 60×, 65×, 70×, or 75× the concentration of the platelets in whole blood. In some embodiments, the concentration of nucleated cells is at least or at least about 5×, 10×, 15×, 20×, 25×, 30×, 35×, 40×, 45×, 50×, 55×, 60×, 65×, 70×, or 75× the concentration of the nucleated cells in whole blood. In some embodiments, the concentration of monocytes is at least or at least about 5×, 10×, 15×, 20×, 25×, 30×, 35×, 40×, 45×, 50×, 55×, 60×, 65×, 70×, or 75× the concentration of the monocytes in whole blood. In some embodiments, the concentration of granulocytes is at least or at least about 5×, 10×, 15×, 20×, 25×, 30×, 35×, 40×, 45×, 50×, 55×, 60×, 65×, 70×, or 75× the concentration of the granulocytes in whole blood.

In some embodiments, the concentration of the platelets, the nucleated cells, the monocytes, the lymphocytes, the granulocytes, or a combination thereof is each at least 10× the concentration in whole blood. In some embodiments, the concentration of platelets is at least or at least about 10× the concentration of the platelets in whole blood. In some embodiments, the concentration of nucleated cells is at least or at least about 10× the concentration of the nucleated cells in whole blood. In some embodiments, the concentration of monocytes is at least or at least about 10× the concentration of the monocytes in whole blood. In some embodiments, the concentration of granulocytes is at least or at least about 10× the concentration of the granulocytes in whole blood. In some embodiments, the concentration of platelets is at least or at least about 10× the concentration of the platelets in whole blood and the concentration of nucleated cells is at least or at least about 10× the concentration of the nucleated cells in whole blood. In some embodiments, the concentration of platelets is at least or at least about 10× the concentration of the platelets in whole blood and the concentration of nucleated cells is at least or at least about 5× the concentration of the nucleated cells in whole blood.

In some embodiments, the composition is for use in treating, preventing, or ameliorating a tissue disease; regenerating tissue; reducing bacterial dysbiosis in a tissue; or a combination thereof.

In some embodiments, the tissue is avascular tissue. In some embodiments, the tissue is intervertebral disc tissue. In some embodiments, the avascular tissue is an intervertebral disc. In some embodiments, the tissue is more than one intervertebral disc. In some embodiments, the tissue is already damaged. In some embodiments, the tissue may be at risk of becoming damaged.

In yet another aspect, the present disclosure provides a method of making the platelet-rich plasma composition (the composition comprising: plasma; platelets at a concentration of at least or at least about 5× to at least or at least about 75× the concentration of platelets in whole blood; and nucleated cells at a concentration of at least or at least about 5× to at least or at least about 75× the concentration of nucleated cells in whole blood, wherein the nucleated cells comprise monocytes, lymphocytes, granulocytes, or a combination thereof), the method comprising the steps of: (a) centrifuging blood in a first vial or container, thereby producing a first upper fraction comprising plasma, platelets, and nucleated cells; (b) decanting or transferring the first upper fraction from the first container into a second vial or container; (c) centrifuging the first upper fraction in the second vial or container, thereby producing a second upper fraction comprising plasma; and (d) reducing the volume of plasma in the second upper fraction.

In some embodiments, the first upper fraction is substantially free of red blood cells.

In some embodiments, the step of decanting or transferring the first upper fraction comprises manual decanting.

In some embodiments, the second upper fraction is substantially free of platelets and nucleated cells.

In some embodiments, the step of reducing the volume of plasma in the second upper fraction comprises manual decanting or aspiration by pipette.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows magnetic resonance imaging (MRI) images of a patient demonstrating the “degenerative cascade.” The MRI images on the left are T2 sagittal (T2-weighted, sagittal plane) images of the lumbar spine and on the right are T2 axial (axial plane) images through the L4-5 level (lumbar vertebrae 4-5 and the L4-5 disc). The two MRI images on the top are from 2013. The two MRI images on the bottom are from 2021. The L4-5 disc has completely degenerated over time, causing progressive narrowing of the spinal canal (spinal stenosis).

FIG. 2 shows fluoroscopic images. On the left, the needles are placed into the two lower discs (L4-5 and L5-sacral vertebra 1 (S1)) without contrast injection. The images on the right are after contrast injection (black fluid inside the disc). In a normal discogram, the contrast should stay in the center of the disc (the nucleus pulposus) and not leak out (L4-5 is normal). In the lower disc, there is a faint black line of contrast (arrow) outlining the back of the disc that represents a tear of that disc (L5-S1). If the patient experiences similar pain to what they normally have (what is referred to as concordant pain) when that tear fills with contrast, then that is an abnormal discogram. The discogram helps confirm the diagnosis of internal disc disruption when the MRI is inconclusive. In this study, patients were then randomized to receive either more contrast or leukocyte-rich platelet-rich plasma (LR-PRP).

FIG. 3 shows magnified MRI images of lumbar spine from a side view (T2 sagittal images). The image on the left is before PRP treatment and shows a white line in the back of the disc called a high-intensity zone (HIZ). The HIZ on the MRI represents a tear inside the disc. The image on the right shows the HIZ healed 3 months after PRP treatment.

FIG. 4 shows magnified T2 sagittal and axial MRI images of lumbar spine from a patient who received intradiscal LR-PRP treatment. The images on top are before treatment and the images on the bottom are from 3 months after LR-PRP treatment. The images reveal structural improvement in the large annular fissure and a reduction in the size of the left-sided protrusion.

FIG. 5 shows the change in C. Acnes survival over time when cultured in LR-PRP vs. leukocyte-poor platelet-rich plasma (LP-PRP). LR-PRP created a greater degree of suppression of C. acnes growth in vitro.

FIG. 6 shows hemocytometer measurements (blood count histograms) of baseline peripheral blood and final LR-PRP distributions of white blood cells (WBC), red blood cells (RBC), and platelets (PLT). The x-axis reflects cell size. See Table 1.

FIG. 7 shows T2 sagittal MRI images pre- and post-intradiscal LR-PRP demonstrating improvement in the Modic Type 1 changes (MC1).

FIGS. 8A-8C show fluoroscopic images, captured at different stages and angles, of an intradiscal insertion of a curved catheter to precisely deliver LR-PRP into the right posterolateral corner of the L4-5 disc.

FIG. 9 shows T2 sagittal and axial MRI images from 2013 revealing an HIZ and a small protrusion effacing the right LA nerve root.

FIG. 10 shows serial T2 axial MRI images over time demonstrating structural changes of the right-sided annular fissure and protrusion at one, two, and three months post-intradiscal LR-PRP precisely delivered with a curved catheter.

FIG. 11 shows a magnified T2 axial MRI image of right foraminal HIZ demonstrating structural changes at 1 month post-intradiscal LR-PRP delivered precisely into the region with a curved intradiscal catheter.

FIG. 12 shows CT-discogram and T2 sagittal MRI images. Images on top are CT-discogram axial and sagittal images of significant annular disruption of the L4-5 disc in a patient later treated with intradiscal LR-PRP. Images below are T2 sagittal MRI images from the same patient, before treatment and 11 years after treatment, showing no signs of disc degeneration at the treated L4-5 level.

FIG. 13 shows a representative magnetic resonance imaging (MRI). Panel (A) is an MRI sagittal T2 image of Modic Type 1 changes at L4-5 and L5-S1 pre-treatment. Panel (B) shows lateral fluoroscopic images of intradiscal injection of contrast at L4-5 and L5-S1 showing annular fissures on the left and post-intradiscal leukocyte-rich platelet-rich plasma injections on the right. Panel (C) is an MRI sagittal T2 image of improvement at L4-5 and L5-S1 at 1 year post-treatment.

FIG. 14 illustrates an example density gradient of blood fractionation. The relative density of blood components is measured in g/mL.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In various cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting.

### Introduction

Platelet-rich plasma (PRP) has been used for decades for various purposes. The term “platelet-rich plasma” is a very general term used to describe a fraction created after centrifuging blood. This blood is usually obtained from the patient being treated. The term is general such that there are significant differences in the various PRP compositions made by different commercial devices. For example, the platelet and cellular content of some PRP formulations have higher red cell and white cell cellular content and other PRP formulations are mostly devoid of white cells and red cells. These differences are mostly artifacts of the commercial system used to make the PRP and are not related to any clinical decision. For example, the ARTHREX® ANGEL® System produces PRP that has a higher hematocrit and white cell content than REGENLAB® PRP®.

The potency of PRP is often measured by the increase of certain captured blood fractions over their respective levels in whole blood. For example, PRP with a concentration of 400,000 platelets per microliter, which was made from a blood draw with 200,000 platelets per microliter of blood, would be said to have a 2× increase in platelets. This same metric could be used to describe other components of blood found in PRP such as granulocytes, lymphocytes, and monocytes.

The clinical use of PRP ranges from treating conditions such as hair loss, wrinkle removal, arthritis, pain management, and a wide variety of musculoskeletal conditions. Mixed results are documented in studies using different formulations of PRP to reduce inflammation and/or repair damaged tissue due to age or injury. This mixed data set is not surprising given the broad range of conditions studied and the dramatic difference in the cellular components of the PRP used in the studies. PRP therapy is typically not covered by insurance because insurance companies deem it as experimental.

Generally, a theory supporting the therapeutic use of PRP is that the cells and proteins contained in the PRP composition support the formation of new blood vessels with the resulting improved blood supply contributing toward the repair of the tissue. This theory is different, however, when treating tissue that does not rely on a blood supply for perfusion. For example, cartilage on the end of long bone and the intervertebral disc (IVD) do not have an intrinsic blood supply. These tissues rely on the movement of fluid based on changes in pressure caused by shifting weight. When a person is up and exercising, the fluid moves from the tissue into the joint space. When a person is resting, fluid seeps back into the tissue. Establishing a blood supply in this tissue would be unhelpful. Consequently, clinicians prefer to use a PRP that is devoid of white cells and red cells in the joint space for patients with osteoarthritis (OA). Often clinicians will then default to a PRP system that removes virtually all of these cell fractions. Without being bound by any theory, the current understanding of this practice is that the platelets, not the white cells, release transforming growth factor beta (TGF-β) proteins that act as a long-term anti-inflammatory and pro-healing agent.

Recently, the potential role that certain bacteria play in causing intervertebral disc degeneration (IVDD) have been investigated. Recent studies have suggested a “gut-disc axis” and have put into question the sterility of even normal discs. Studies have evaluated the microbiome of normal versus degenerated discs and found that even discs appearing normal on magnetic resonance imaging (MRI) had greater than 50 bacterial species. Further analysis has found an overgrowth (dysbiosis) of certain types of bacteria (Pseudomonas and Streptococcus) in degenerated discs. There is now mounting scientific evidence that bacteria play a greater role in IVDD than previously recognized. Once these occult infections take hold in the disc, the person feels pain and the disc begins to deteriorate.

IVDD dysbiosis is now believed to be an important cause of back pain, inflammation, and degeneration. See, for example, Gilligan et al. (2021). It is believed that the overgrowth of bacteria upregulates pro-inflammatory cytokines that are commonly associated with IVDD. This disease progression is often called Modic Type 1 changes (MC1) and is typically diagnosed through MRI imaging. The end of the process often leads to an invasive surgical spine fusion because conservative treatments have failed to address the underlying pathophysiology.

This new information has paradigm-shifting effects on new novel potential therapies for IVDD and may explain why traditional treatments of drugs and surgery often fall short of offering patients with chronic lower back pain a long-term solution. Some researchers have advocated for a prolonged course of oral antibiotics (100 days) to treat IVDD, but clinical results have been mixed. This would be poor antibiotic stewardship and may result in the emergence of antibiotic resistant bacteria. Additionally, the disc is the largest avascular structure in the human body, so the actual penetration of oral antibiotics that rely on a blood supply for perfusion would be variable.

Some patients opt for invasive surgical intervention, which is highly susceptible to infections due to the fact that implants are being placed into an environment that already has bacteria present. Often the bacteria adhere to the foreign substance that the implant is made from such as the screw, the interbody device, or the plate. The bacteria then create a biofilm over the implant that leads to chronic pain and has now become the leading causes of implant loosening and spinal fusion failure. This may explain why up to 60% of patients are still taking opioid pain medications 3 months after spinal fusion. Antibiotics delivered by a traditional route that relies on a blood supply to reach the area of infection are not effective in these cases because of this biofilm on the inert implant, and revision surgery is often performed to remove the spinal implants so that the infection can be treated.

One of the novel features of the present disclosure is the discovery of these occult infections inside the intervertebral disc (IVD) and the role that a certain type of platelet-rich plasma (PRP) preparation would have on its treatment. Interestingly, this novel insight has led to the novel treatment protocol disclosed herein. Rather than focus on the anti-inflammatory formulations of PRP that have a low concentration of white cells (leukocytes) and red cells (erythrocytes), results disclosed herein found that the opposite formulation was beneficial. By going against the teachings of the general body of literature, it is disclosed herein that the infection-fighting component of a concentrated blood fraction that is rich in white cells is very beneficial in tissue where such non-systemic local infections reside. The active component in the treating composition is not just the platelets, but also the white cells.

By designing a unique blood concentration device and implementing the protocols, compositions, and methods disclosed herein, it was discovered that leukocyte-rich platelet-rich plasma (LR-PRP) was much more effective in these infected tissues than leukocyte-poor PRP. To create this treating composition included a specific protocol that involved a centrifugation regimen that targeted not only the platelets, but the granulocytes. The protocol included a first centrifugation step, a manual decanting of the upper fraction of blood to include the majority of the leukocytes, and then then a second centrifugation step. Other centrifugation protocols are contemplated. Additionally, the concentration of the leukocytes was found to be 5× or 10× greater than the blood draw (whole blood). The method included delivering the LR-PRP intradiscally through an injection procedure to allow it to be in the general area of the infection. In addition, combining the LR-PRP with an antibiotic delivered intradiscally so that it would not rely on the circulatory system would have additional benefit.

The treating composition contemplates using a concentrate of the components of blood based on a density gradient separation (see, e.g., FIG. 14). The treating composition will generally encompass components, including cells and platelets, that range from 1.06 g/mL to 1.08 g/mL. The concentrate will include an increase over the whole blood levels of these components in the range of 5×, 10×, 20×, or greater. A novel feature of this disclosure is that these highly concentrated components include leukocytes and will repair the disc. It has been discovered that, in some embodiments, concentrations as high as 70× or higher are desirable. Unlike in other tissues, such as intestine, the higher dose of cells and platelets did not have a diminishing benefit or even a harmful side effect in IVD tissue. This insight is contrary to conventional thinking.

### Definitions

### General

As used herein, the indefinite articles “a,” “an,” and “the” should be understood to include plural reference unless the context clearly indicates otherwise. When introducing elements disclosed herein, the articles “a,” “an,” “the,” and “said” are intended to mean that there are one or more of the elements. Further, the one or more elements may be the same or different. Thus, for example, unless the context clearly indicates otherwise, “an agent” includes a single agent, and two or more agents. Further, the two or more agents can be the same or different.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or step. When used herein, the term “comprising” can be substituted with the term “containing” or “including.”

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours. As used herein, the symbol “±” denotes a range, that is, where a given value is “N±X” a range from N−X to N+X, wherein the range includes the values of N−X and N+X.

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, “increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99, or 100%, for example, as compared to the level of a reference, standard, or control.

As used herein, “increases” also means increases by at least 1-fold, for example, 1-, 2-, 3-, 4-, 5-, 6-, 7-, 8-, 9-, 10-, 15-, 20-, 30-, 40-, 50-, 60-, 70-, 80-, 90-, 100-, 200-, 500-, or 1000-fold or more, for example, as compared to the level of a as compared to the level of a reference, standard, or control.

As used herein, “decreasing” refers to decreasing by at least 5%, for example, 5, 6, 7, 8, 9, 10, 15, 20, 25, 30, 35, 40, 45, 50, 55, 60, 65, 70, 75, 80, 85, 90, 95, 99, or 100%, for example, as compared to the level of a reference, standard, or control.

As used herein, “decreases” also means decreases by at least 1-fold, for example, 1-, 2-, 3-, 4-, 5-, 6-, 7-, 8-, 9-, 10-, 15-, 20-, 30-, 40-, 50-, 60-, 70-, 80-, 90-, 100-, 200-, 500-, or 1000-fold or more, for example, as compared to the level of a reference, standard, or control.

As used herein, the term “reference” means a standard or control condition (e.g., untreated with a test agent or combination of test agents). Alternatively, “reference” may refer to a resource, such as an annotated genome, transcriptome, or the like, that is used to assemble, analyze, and/or interpret data.

As used herein, the term “eliminate” means to decrease to a level that is undetectable.

### Subjects

The terms “subject” and “patient” are used interchangeably herein. The term “patient” refers to a human, while the term “subject” may refer to a human or a non-human animal.

The term “subject” refers to a vertebrate, including any member of the class Mammalia. As used herein, “subject” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a subject is a human. In still further embodiments, a subject of the disclosure may be a cell, cell culture, tissue, organ, or organ system.

In some embodiments, a subject is a human female. In some embodiments, a subject is a human male. In some embodiments, a subject is an intersex human. In some embodiments, a human subject has physiological and/or genetic characteristics associated with one or more sexes; has undergone or received medical interventions that affect physiological characteristics associated with one or more sexes; and/or is intersex. In some embodiments, the sex of a subject is undefined, unknown, or unclear.

In some embodiments, the subject is at least about 1 month of age, for example, at least about 2, 3, 4, 5, 6, 7, 8, 9, 10, 11, 12, 15, 18, or 21 months of age, or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years of age. In some embodiments, the subject is about 1-100, 1-80, 1-60, 1-30, 1-24, 1-20, 1-18, 1-12, 1-10, 1-8, 1-6, 2-100, 2-80, 2-60, 2-30, 2-24, 2-20, 2-18, 2-12, 2-10, 2-8, 2-6, 3-100, 3-80, 3-60, 3-30, 3-24, 3-20, 3-18, 3-12, 3-10, 3-8, 3-6, 4-100, 4-80, 4-60, 4-30, 4-24, 4-20, 4-18, 4-12, 4-10, 4-8, 4-6, 5-100, 5-80, 5-60, 5-30, 5-24, 5-20, 5-18, 5-12, 5-10, 5-8, 6-100, 6-80, 6-60, 6-30, 6-24, 6-20, 6-18, 6-12, 6-10, 8-100, 8-80, 8-60, 8-30, 8-24, 8-20, 8-18, 8-12, 10-100, 10-80, 10-60, 10-30, 10-24, 10-20, 10-18, 12-100, 12-80, 12-38, 12-60, 12-50, 12-40, 12-30, 12-24, 12-20, 12-18, 18-100, 18-80, 18-60, 18-50, 18-40, 18-30, 18-24, 20-100, 20-80, 20-60, 20-50, 20-40, 20-30, 20-25, 30-100, 30-80, 30-60, 30-55, 30-50, 30-45, 30-40, 40-100, 40-80, 40-60, 40-55, or 40-50 years of age. In some embodiments, the subject is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, or 100 years of age. In some embodiments, the subject is about 14-72 years of age. In other embodiments, the subject is a fetus. In some embodiments, the subject is a neonatal subject.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older, 75 years of age or older, or 90 years of age or older. In some embodiments, the subject is 50 years of age or older. In some embodiments, the subject is a child. In some embodiments, the subject is 18 years of age or

nger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age, or 12-18 years of age.

In some embodiments, the subject is about 2-11, 4-17, 12-18, 18-50, 18-90, or 50-90 years of age.

### Treatments & Compositions

In some embodiments, the disclosure provides for methods of treatment. As used herein, “therapy,” “treat,” “treating,” or “treatment” means inhibiting or relieving a condition in a subject in need thereof. For example, a therapy or treatment refers to any of: (i) the prevention of symptoms associated with a disease or disorder; (ii) the postponement of development of the symptoms associated with a disease or disorder; and/or (iii) the reduction in the severity of such symptoms that will, or are expected, to develop with said disease or disorder. The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment. It will be appreciated that, although not precluded, treating a disorder or condition does not require that the disorder, condition, or symptoms associated therewith be completely eliminated.

As used herein, the term “effective amount” or “therapeutically effective amount” means an amount of a composition that, when administered alone or in combination to a cell, tissue, or subject, is effective to achieve the desired therapy or treatment under the conditions of administration. For example, an effective amount is one that would be sufficient to bring about effectiveness of a therapy or treatment. The effectiveness of a therapy or treatment (e.g., eliciting regeneration or repair of a tissue, reducing or preventing degeneration of a tissue, reducing pain) can be determined by suitable methods known in the art. In some embodiments, “regeneration” refers to cellular and/or tissue growth or re-growth.

“Administering” or “administration” as used herein refers to taking steps to deliver an agent to a subject, such as a mammal, in need thereof. Administering can be performed, for example, once, a plurality of times, and/or over one or more extended periods. Administration includes both direct administration and indirect administration, including an act of prescribing a drug or directing a subject to obtain treatment with an agent. For example, as used herein, one (e.g., a physician) who instructs a subject (e.g., a patient) to self-administer or obtain treatment with an agent (e.g., a drug), or to have an agent administered by another and/or who provides a patient with a prescription for a drug is administering an agent to a subject. Administration of an agent can be once in a day or more than once in a day (e.g., twice a day or more). Administration of the agent can be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose.

In some aspects of the disclosure, treatment is in combination with known treatments. In some embodiments, a method disclosed herein comprises administering to the subject two or more agents, for example, 2, 3, 4, or 5 or more agents. In some embodiments, the two or more agents are administered together. In other embodiments, the two or more agents are administered separately, e.g., sequentially. In some embodiments, two or more agents are administered in the same composition. In some embodiments, two or more agents are administered in different compositions.

### Compositions

As used herein, the term “isolated” refers to a material that is free to varying degrees from components which normally accompany it as found in its original or natural state. “Isolate” denotes a degree of separation from original source or surroundings. In some embodiments, an isolated material is considered to the substantially free of other components.

As used herein, the term “nucleated cells” refers to cells having a nucleus that are usually found in whole blood. Examples of nucleated cells include, but are not limited to, leukocytes (white blood cells), leukocyte precursors, and cells descended or derived from leukocytes. Examples of leukocytes include monocytes, lymphocytes, and granulocytes. In some embodiments, lymphocytes are recited as an archetypal leukocyte. Examples of lymphocytes include B cells and T cells, including plasma cells and cytotoxic T cells. Examples of granulocytes include neutrophils, eosinophils, and basophils. In some embodiments, neutrophils are recited as an archetypal granulocyte. In some embodiments, nucleated cells include nucleated red blood cells (erythrocyte or red blood cell precursors) and/or other circulating cells.

### EXEMPLIFICATION

### EXAMPLE 1

A disclosure similar to the following example is published as “Intradiscal Leukocyte Rich Platelet Rich Plasma for Degenerative Disc Disease” in Physical Medicine and Rehabilitation Clinics of North America (Lutz 2022), the contents of which are herein incorporated in their entirety by reference.

There is mounting scientific evidence that chronic low back pain is an unhealed wound inside the disc that frequently gets infected. When the disc tears, it can become infected with a certain type of bacteria that impedes the healing process further called Cutibacterium Acnes (C. Acnes). Proper wound healing includes the migration of cells to the wound. Since the disc is the largest avascular structure in the body, its inherent capacity to heal after injury is poor. The low blood supply to the disc is also why most medical and surgical treatments for it fail to provide sustained relief. They are not addressing the underlying root causes of degenerative disc disease (DDD). When a disc develops a tear that does not heal, the wound can continue to propagate, causing the disc to bulge. The bulge then begins to protrude. If not addressed, the protrusion then can go on to a disc extrusion or even a disc fragment in the spinal canal. By this stage, the disc has degenerated, and that segment of the spine can no longer function properly. Loading of that spinal segment begins to shift elsewhere. The downward spiral can continue to get worse from here, and this is what we refer to as the “degenerative disc cascade.” The degenerative cascade demonstrates the importance of acting early with a regenerative treatment that heals the disc so that this downward spiral can be avoided. Wound healing is a dynamic process for restoring the normal architecture and functionality of tissue. The downward spiral can continue to get worse from here, and this is what is referred to as the “degenerative disc cascade.” The degenerative disc cascade demonstrates the importance of acting early with a regenerative treatment that heals the disc so that this downward spiral can potentially be prevented.

### Introduction

Degenerative disc disease (DDD) is the number one cause of chronic low back pain (CLBP). According to the numbers, low back pain is a pandemic; a condition that is prevalent globally, affecting millions of people throughout the world. In 2017 the Global Burden of Disease (GBD) study reported that the point prevalence (the number of people in the world at one point in time) of activity-limiting lower back pain about 580 million people worldwide-and chronic, low back pain is now regarded as the number one cause of disability globally (James et al. 2018). This is the most comprehensive analysis of 354 medical conditions from 195 countries for nearly three decades from 1990 to 2017. Not only was CLBP the recent number one cause of years lived with disability (YLDs), it has been the number one cause of YLDs every year since 1990 and its incidence is only increasing with time.

The numbers reported are staggering in the United States (U.S.): as many as seventy million Americans have chronic lower back pain today. An estimated 80% of all Americans will experience disabling lower back pain at some point in their lifetime. What was thought of as a benign condition really is not. Many patients have chronic recurrent episodes of lower back pain (LBP) that just keep getting worse over time. In one study from 2012, researchers surveyed thirty different practitioners from a variety of specialties including physical therapists, chiropractors, and surgeons to look at the disease progression of CLBP in 600 new patients (Donelson et al. 2012). Here's what they found:

35% said their back pain required more than 3 months to improve.

54% reported more than ten episodes of severe, disabling back pain.

20% reported more than fifty episodes of severe, disabling back pain.

An epidemic is a condition that affects a large number of people within a region, and the U.S. has been dealing with an opioid epidemic. Mismanagement of LBP has only contributed to this problem; in the U.S., opioids are the most commonly prescribed drug class for patients with LBP and the rates of opioid prescribing is two to three times higher than in most European countries (Deyo et al. 2015). This is despite evidence that opioids, if they are to be used for managing LBP, should only be used for acute pain and for a short duration of time (a few days at most). There are no studies that support the long-term use of opioids for managing LBP.

Complications of addiction and overdose have risen in parallel with increased prescription rates for LBP. According to the Centers for Disease Control and Prevention (CDC), from 1999 to 2019, nearly 500,000 people died from opioid overdose involving both illicit and prescription uses (Scholl et al. 2018). More than 11.5 million Americans reported misusing prescription opioids in 2016 (Centers for Disease Control and Prevention 2018). In 2019, more than half of all global overdose deaths occurred in the U.S. (Vos et al. 2020). Unfortunately, this trend has only increased; last year overdose deaths from drug overdoses hit an all-time high at over 100,000 from both illicit and prescription-based causes. While there are many factors that contributed to these numbers, the liberal prescribing of opioids for patients with CLBP has contributed greatly to this public health crisis. The need for opioid-sparing treatments for patients with CLBP has never been greater.

Many patients with complications from spinal surgery had never previously taken opioids but were started on them at the time of their surgery to manage their post-operative pain. Failing to achieve adequate pain relief from their surgery, their providers just kept prescribing opioid medications month after month. In a recent meta-analysis of patients after lumbar fusion surgery, investigators found that up to 63% of these patients were on long-term opioids (for >3 months) (Lo et al. 2020).

This study also showed that opioid-naive patients were at increased risk for long-term opioid use after their fusion surgery. Despite these poor outcomes, the number of these types of lumbar fusions for degenerative conditions has increased, according to one study, 276% from 2002 to 2014 (Deng et al. 2021). Millions of people suffer from their CLBP who then become dependent on opioids because their treatment has been mismanaged with ineffectual nonsurgical treatments or potentially made worse with surgery.

Drugs and surgery have not been the cure for the majority of patients with CLBP. The economic consequences are not just significant for the individual who fails to get back to gainful employment, but also for the healthcare system as a whole. In 2011, the respected National Academy of Medicine estimated that just in the U.S., the total direct and indirect costs of managing chronic pain from all musculoskeletal conditions to the economy ranges between $560 and $630 billion annually (National Academies of Sciences, Engineering, and Medicine 2011).

Unfortunately, there has been low investment in research to find a cure for CLBP because many are unaware of its severity as most people do not die from it. The U.S. National Institute of Health budget for research on cardiovascular diseases and cancer is dramatically larger than its budget for musculoskeletal (MSK) conditions ($8.6 billion combined vs. only $754 million in 2018). In 2016, MSK disorders were the largest health expenditures in the U.S. at $380 billion (Dieleman et al. 2020). More money is spent treating MSK conditions than heart disease, diabetes, or cancer, but less than 10% of research dollars are allocated to finding a cure for them.

The economic consequences of this irrational strategy are significant not just for the U.S., but for other countries around the world. The increased burden of non-fatal diseases such as CLBP on healthcare systems worldwide is posing considerable challenges to all healthcare systems not equipped to care for such complex and expensive conditions. While healthcare systems have made advancements in the management of fatal diseases, they have not made meaningful advancements in managing “non-fatal” conditions such as CLBP.

The root causes of CLBP should to be reconsidered and treatments targeted directly at them to truly find a cure. Simple, safe, cost-effective, scalable treatments that are durable in their effect are needed. Healthcare systems should to shift their approach to musculoskeletal diseases away from volume-based palliative treatments to value-based root cause treatments that create sustained improvements. As disclosed herein, intradiscal leukocyte-rich platelet-rich plasma (LR-PRP) has the potential to be a value-based root cause treatment for many patients with symptomatic lumbar disc disease.

### Getting to the Root Causes of Degenerative Disc Disease

There is mounting scientific evidence that chronic low back pain is an unhealed wound inside the disc that frequently gets infected. When the disc tears, it can become infected with a certain type of bacteria that impedes the healing process, Cutibacterium Acnes (C. Acnes) (Gilligan et al. 2021). Proper wound healing includes the migration of cells to the wound. Since the disc is the largest avascular structure in the body, its inherent capacity to heal after injury is poor.

The low blood supply to the disc is also why most medical and surgical treatments for it fail to provide sustained relief. They are not addressing the underlying root causes of degenerative disc disease (DDD) (Grunhagen et al. 2006). When a disc develops a tear that does not heal, the wound can continue to propagate, causing the disc to bulge. The bulge then begins to protrude and, if not addressed, the protrusion then can go on to become a disc extrusion or even a disc fragment in the spinal canal. By this stage, the disc has degenerated and that segment of the spine can no longer function properly. Loading of that spinal segment begins to shift elsewhere.

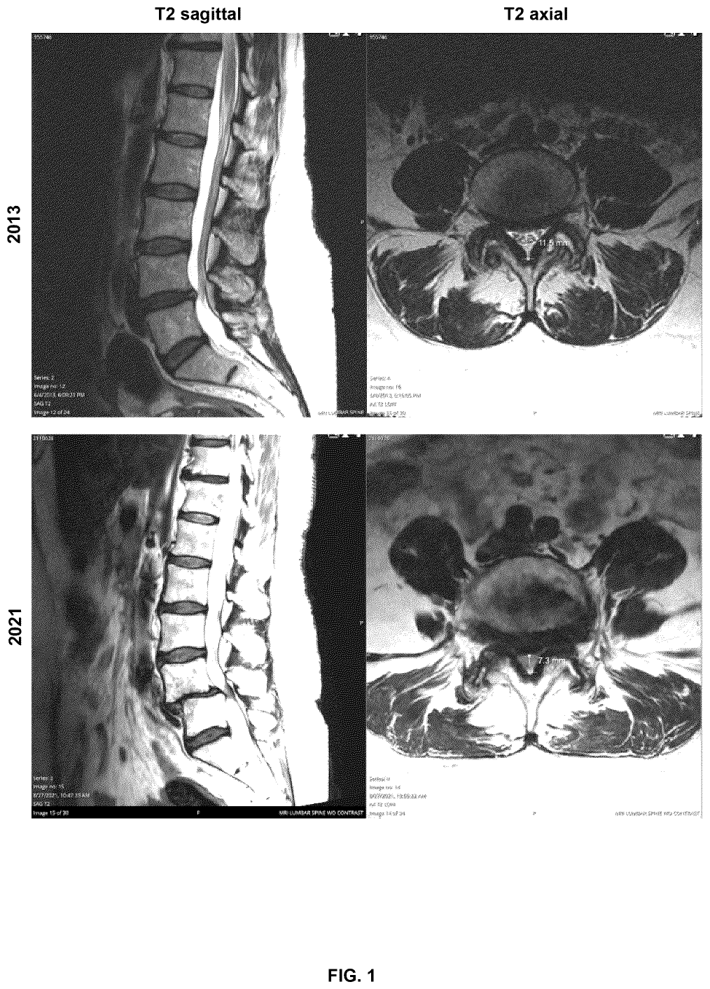

The downward spiral can continue to get worse from here; this is the “degenerative disc cascade” (FIG. 1) (Kirkaldy-Willis et al. 1976, Kho et al. 2018). The degenerative cascade demonstrates the importance of acting early with a regenerative treatment that heals the disc so that this downward spiral can be avoided. Wound healing is a dynamic process for restoring the normal architecture and functionality of tissue.

### The Role Bacteria Play in Degenerative Disc Disease

A microbiome is an environment of trillions of microorganisms also called microbiota or microbes (Albert et al. 2013). These can be a collection of thousands of different species of bacteria, fungi, parasites, and/or viruses that live in harmony when a subject is healthy, but when out of balance can be harmful. This is referred to as “dysbiosis.” Scientists are just realizing the importance of the microbiome not only for overall health but also with regard to degenerative disc disease (DDD).

A recent study out of Sweden looked at the role the intradiscal microbiome might play in degenerative disc disease (Chen et al. 2016). The study examined 162 patients who experienced chronic low back pain and had Modic Type 1 changes (MC1) in their magnetic resonance imaging (MRI). In a double-blind randomized controlled study (DB RCT), they separated the patients into two groups and gave one group a 6-month course of oral antibiotics and the other group a placebo. The subset of patients that received the antibiotics improved to a greater degree than the control group. Their findings suggested bacteria may be playing a greater role in the pain caused by degenerative disc disease than previously realized.

This issue was also studied in patients having spinal surgery where they harvested disc material, whether from a herniation or degeneration and cultured that material to see if any bacteria would grow. Study after study showed that bacteria grew on the disc material and the most common bacteria was C. Acnes. At first, investigators thought that this was from contamination, but that has been refuted. The presence of C. Acnes infecting extruded and degenerative discs has been unequivocally demonstrated now by more sophisticated testing measures.

In 2016, a group of researchers in China took C. Acnes harvested from human disc surgical samples and injected it into rabbit discs to see its effects (Akeda et al. 2006). When they examined the discs by MRI and under the microscope weeks later, these injected discs demonstrated exactly the same findings as with degenerative disc disease and MC1.

There are a number of valid reasons why CLBP patients have not been treated with oral antibiotics:

1. The question of whether or not bacteria in the disc represents infection or contamination

2. Contradictory reports on the efficacy of antibiotics to treat CLBP patients

3. The potential that widespread use of systemic antibiotics would result in emerging global antimicrobial resistance and the perceived risk of propagating superbugs

4. Systemic antibiotics can adversely affect the gut's microbiome creating other negative health consequences. Patients in these studies had to take the antibiotic for 100 days

5. The penetration of oral antibiotics that rely on blood flow to get to the disc is unreliable

If CLBP is viewed as an unhealed wound inside the disc that often becomes contaminated with bacteria, it can be understood how previous treatments have been off the mark. Without being bound by any theory, with the leukocyte-rich platelet-rich plasma (LR-PRP) disclosed herein, billions of platelets with thousands of growth factors were injected into the disc tissue to stimulate the wound healing process; millions of white blood cells were also injected, which may also suppress the overgrowth of harmful C. Acnes or other bacteria that penetrated those tears.

### A Regenerative Medicine Solution for Degenerative Disc Disease

According to the National Institutes of Health (NIH), regenerative medicine is an emerging area of science that holds great promise for treating and even curing a variety of injuries and diseases by using stem cells and other technologies to repair or replace damaged cells, tissues, or organs. However, there are many healing cells and proteins in the body that can stimulate natural healing processes. Regenerative medicine offers not only the hope of a cure for degenerative disc disease (DDD), but also a potentially sustainable solution to the most common, most expensive, and most disabling condition globally: DDD. While there are many clinicians investigating a variety of intradiscal biologics, leukocyte-rich platelet-rich plasma (LR-PRP) has the most compelling scientific evidence. According to the present disclosure, it has also been a safe and effective intradiscal biologic for chronic low back pain (CLBP) patients.

In 2006, researchers at Rush Medical College in Chicago were the first to show that platelet-rich plasma (PRP) had a beneficial effect on stimulating disc cells (Chen et al. 2006). Scientists made PRP from pig's blood, cultured disc cells in the lab in a broth of PRP, and measured its effects on cell metabolism. They demonstrated that PRP could stimulate the cells of the disc to turn on and produce collagen. The effect of the PRP was greater on the cells of the annulus fibrosus (AF) than the nucleus pulposus (NP).

At Tapei Medical University in Taiwan, researchers also studied the effects of PRP on human disc cells taken from healthy volunteers (Tuakli-Wosornu et al. 2015). They cultured these cells in PRP and again measured its potential beneficial effects in the lab. Not only were these researchers the first to show the beneficial effects PRP could have on human disc cell metabolism, but they also demonstrated that PRP could decrease the rate of apoptosis. These promising studies showed that PRP caused beneficial effects on cell proliferation (coming to the wound), increased cell metabolism (producing proteins to repair the wound), and decreased cell death (keeping the disc healthy). In addition, researchers also showed that PRP could reduce the number of harmful pro-inflammatory cytokines in the disc that was responsible for pain and degeneration.

According to a clinical study disclosed herein, very strict inclusion and exclusion criteria were set up to specifically study patients suffering from chronic, low back pain as a result of painful annular tears—internal disc disruption (IDD). Other inclusion criteria were: severe disabling back pain that was unresponsive to conservative treatment; and patients who otherwise would be candidates for a spinal fusion.

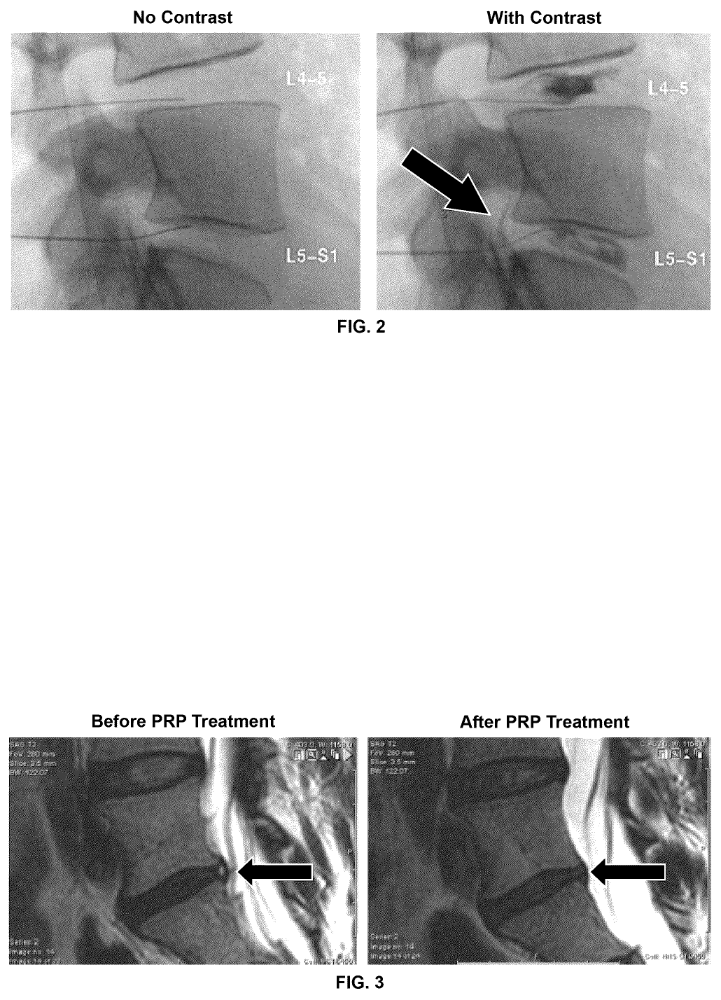

Once selected for the clinical study, a discogram was performed to confirm that the patients' discs were the source of their pain. Patients were then randomized into one of two groups: patients who received PRP after the contrast in the discogram and patients who received a placebo, which was more contrast alone (FIG. 2). Then over an initial period of 2 months, their responses were tracked with an independent observer: degree of pain relief, functional improvement, and patient satisfaction. In total, forty-nine patients were tracked. For every two patients who received the PRP, one patient was in the control group.

A crossover group was added to the clinical study: after 2 months, if the patient did not see improvement, they were unblinded. If they had received the control and had not improved, they were then offered the intradiscal PRP treatment (Monfett et al. 2016). In the first 2 months, patients who received the PRP were showing significant improvements in pain and function, whereas the control group was not. Further, patients who had been in the control group and then unblinded also saw significant improvement when they crossed over into the treatment group and received the PRP. The treated patients were followed for years and, surprisingly, the majority of patients continued to do well from a single intradiscal injection of their PRP (Cheng et al. 2019, Akcda et al. 2019).

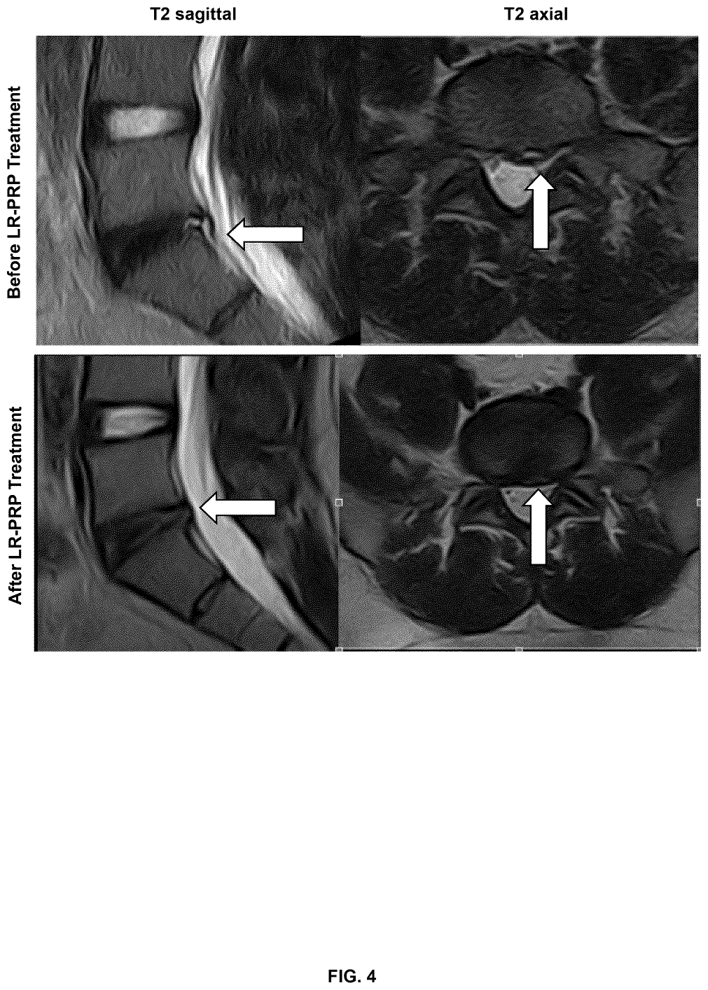

The MRIs of patients that were treated were frequently analyzed. In many of the PRP-treated patients, the tears improved significantly or disappeared within months of treatment (FIG. 3, FIG. 4). Out of the 49 patients enrolled in the study, only six went on to undergo spinal fusion surgery. Intradiscal LR-PRP effectively decreased the surgery rate by roughly 80% in this group of patients.

Since the initial double-blind randomized control study (DB RCT) in 2016, it has been encouraging to witness other investigators from around the world publishing similar encouraging results (Akcda et al. 2022a, Akeda et al. 2022b, Prysak et al. 2021). Akeda and colleagues recently published another DB RCT comparing intradiscal platelet release to corticosteroid injections in patients with degenerative disc disease. While there are several limitations to this study (no control, only 16 patients, corticosteroid injected with 2 mL saline, not an LR-PRP, no statistical difference in outcome), they found that over a 60-week period, the PRP group did experience a greater degree of pain relief and functional improvement. This makes intradiscal PRP the only orthobiologic treatment option with two supportive DB RCT studies.

There was, however, a very recent single-blind randomized controlled study of intradiscal PRP that did not show a significant difference between the treatment group and the control group. However, there were some serious flaws in the methodology that may have confounded the conclusion that intradiscal PRP was no better than the control: they used a leukocyte-poor PRP, they injected only a small amount (1 cc) of a low platelet concentration PRP, they did not quantify what they injected, they did not use contrast to demonstrate flow into the annular tears, they used saline and antibiotics as the control, and they excluded a subset of patients (Modic Type 1 changes) from their study.

According to results disclosed herein, it is exactly that subset of patients that have responded favorably to intradiscal LR-PRP. In addition, using saline and antibiotics are not a negative control. These agents have an antibacterial effect that may have improved some of the patients in the control group, thereby confounding their results. There continues to be a need for more rigorous research on intradiscal PRP for patients with degenerative disc disease.

While overall, the disclosed results achieved with intradiscal LR-PRP were impressive, there were still a fair number of patients that did not improve—roughly 40%.

During the first clinical study, the platelets in the PRP preparation were concentrated to approximately five times the normal baseline concentration. It seemed logical that more platelets would translate into a higher delivery of healing growth factors; however, the hypothesis—whether a greater degree of growth factors could provide pain relief to patients with more severe disc disease—remained to be tested.

Platelet concentrations of greater than ten times the patients' baseline—sometimes even higher—could be achieved on a consistent basis by lowering the volume of plasma (Lutz et al. 2022). The number of patients treated over the past few years with this higher concentration PRP was retrospectively reviewed. It was found that about forty-five patients had received the PRP over a year prior. Data was available on thirty-seven of those patients, which is an acceptable follow-up rate of over 80%. Then their results—pain relief, functional improvement, and patient satisfaction—were compared to the historic DB RCT results to see if they were equal, worse, or potentially better. Not only did were there greater degrees of pain relief and functional improvement, but patient satisfaction rates also reached over 80% (Beatty et al. 2019).

One of the patients in the study had a complication of a spinal infection (Jerome et al. 2021). She was infected with the same bacteria previously discussed—C. Acnes. In an attempt to concentrate the platelets to higher levels, some patients received a leukocyte-poor PRP, and this is what happened in that patient. She had to be treated with a prolonged course of intravenous antibiotics to recover and the infection destroyed that disc.

Leukocyte-Rich PRP: Killing Two Birds with One Stone?

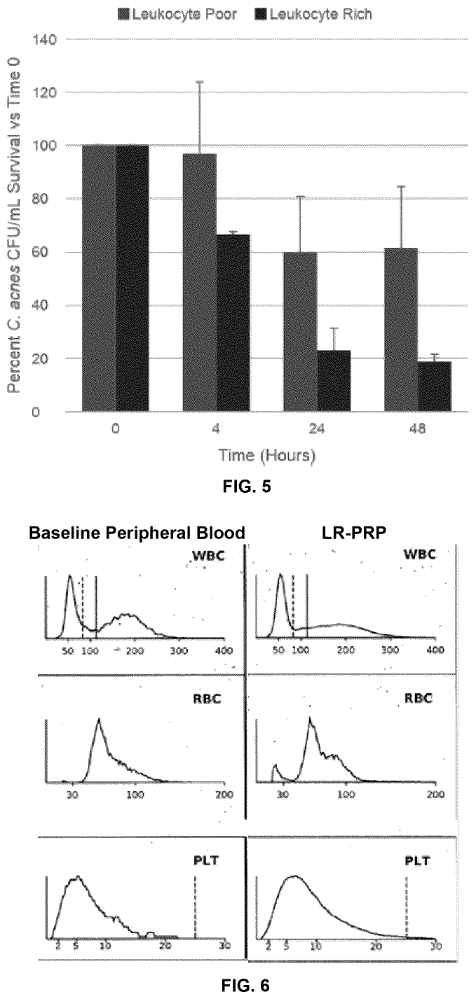

It would be beneficial to reduce the risk so that the potential benefits of this promising new procedure would significantly outweigh the potential risks. C. Acnes was the culprit in many of the infections associated with intradiscal biologic procedures (Prysak et al. 2019). C. Acnes bacteria was cultured in different types of platelet-rich plasma (PRP)—leukocyte-rich (LR) versus leukocyte-poor (LP). The bacteria were cultured for up to 48 hours and it was found that indeed the LR-PRP created greater kill rates than the LP-PRP (FIG. 5) (Jerome et al. 2021, Prysak et al. 2019). These results provided more evidence for not only an effective type of PRP for degenerative disc disease, but a safe type of PRP. The clinical experience disclosed herein reflects this finding. In more than 1000 disc injection procedures, no infections have resulted thus far using an LR-PRP inside the disc.

Two objectives may be accomplished with the intradiscal LR-PRP. Without being bound by any theory, not only is the body's natural healing response in the disc being ignited, but the high levels of white blood cells in the PRP may also be suppressing the overgrowth of certain types of bacteria associated with disc degeneration.

A 32-year-old woman who presented with severe low back pain (LBP) had not responded to traditional treatments. What was unusual about her history was that she could not attribute the onset of her pain to any specific event. She rated the pain as an 8 out of 10. She had a 2-year-old daughter that she was having a difficult time caring for because she could not lift her. An MRI of her lumbar spine was obtained to see what was going on. It revealed two degenerative discs with significant Modic Type 1 changes (MC1).

She had already failed oral medications, chiropractic care, acupuncture, and an epidural steroid injection gave only short-term pain relief. Spinal surgery, she said, was a last resort. After discussing the pros and cons of intradiscal LR-PRP in her case, she agreed to the procedure.

Working with an industry partner, a new PRP system has been developed that is now able to concentrate the platelets and white blood cells to higher levels than an available commercial PRP system on the market (Table 1). Over 5 billion platelets and 100 million white blood cells (WBCs) were injected into each of her discs, delivering thousands of healing proteins in the platelets and the antibacterial power of the WBCs exactly where they were useful. LR-PRP provides a root cause treatment for so many patients with chronic low back pain (CLBP) looking for a better alternative than spinal fusion surgery (FIG. 6).

TABLE 1
Hemocytometer measurements of baseline peripheral
blood and final LR-PRP concentrations.

| | | Normal | Normal | | | | | |
|---|---|---|---|---|---|---|---|---|
| | | Range | Range | Baseline PB | | LR-PRP | | Enrichment |
| | Unit | (Lower) | (Upper) | Value | Flag | Value | Flag | (×) |
| WBC | 10³/mm³ | 4.1 | 10.5 | 3.7 | L | 52.2 | H | 14 |
| LYM | % | 16.0 | 43.3 | 37.7 | | 44.2 | H | |
| MON | % | 2.8 | 10.2 | 7.3 | | 7.3 | | |
| GRA | % | 48.5 | 80.3 | 55.0 | | 48.5 | | |
| LYM | 10³/mm³ | 1.00 | 3.10 | 1.30 | | 23.00 | H | |
| MON | 10³/mm³ | 0.10 | 0.70 | 0.20 | | 3.80 | H | |
| GRA | 10³/mm³ | 2.30 | 7.70 | 2.20 | L | 25.40 | H | 12 |
| RBC | 10⁶/mm³ | 4.30 | 5.80 | 2.54 | L | 2.96 | L | |
| HGB | g/dl | 13.1 | 16.7 | 7.7 | L | 8.6 | L | |
| HCT | % | 39.3 | 51.0 | 22.2 | L | 25.5 | L | |
| MCV | μm³ | 83 | 98 | 87 | | 86 | | |
| MCH | pg | 27.0 | 32.2 | 30.5 | | 28.9 | | |
| MCHC | g/dl | 31.8 | 33.7 | 34.9 | H | 33.6 | | |
| RDW- | % | 11.9 | 14.8 | 13.9 | | 17.4 | H | |
| CV | | | | | | | | |
| PLT | 10³/mm³ | 150 | 399 | 134 | L | 2512 | D | 19 |
| MPV | μm³ | 6.8 | 10.1 | 7.3 | | 8.3 | D | |

WBC, white blood cell; LYM, lymphocyte; MON, monocyte; GRA, granulocyte; RBC, red blood cell; HGB, hemoglobin; HCT, hematocrit; MCV, mean corpuscular volume; MCH, mean corpuscular hemoglobin; MCHC, MCH concentration; RDW-CV, red cell distribution width; PLT, platelet; MPV, mean platelet volume; PB, peripheral blood; LR-PRP, final leukocyte-rich, platelet-rich plasma; L, low; H, high; D, D Flag (Delta Check); ×, fold enrichment.

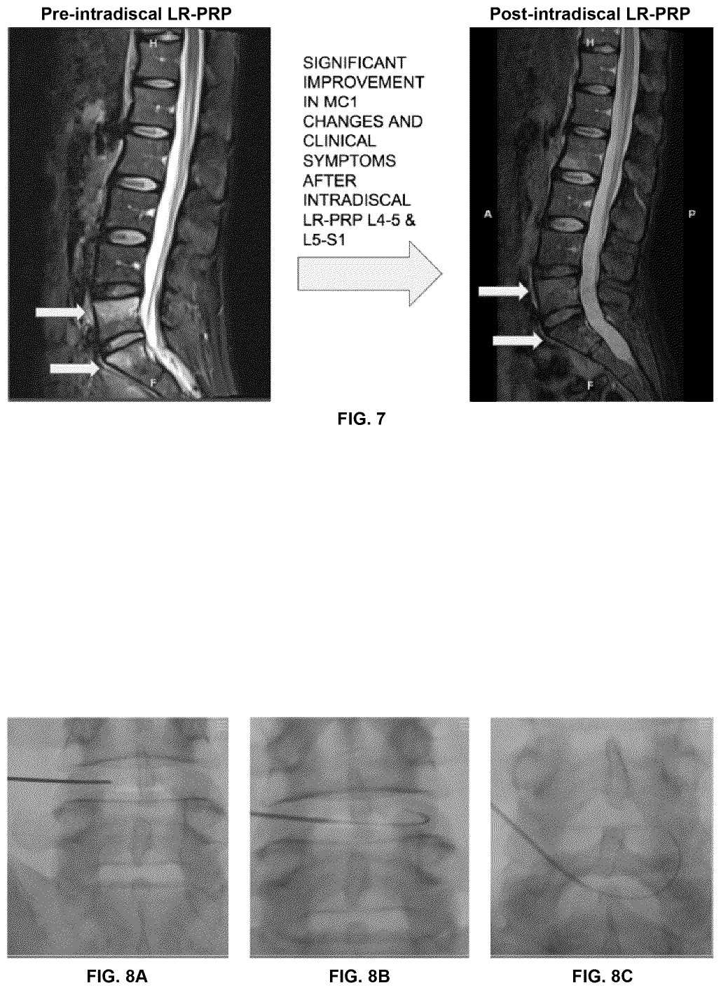

At first, her pain was worse from the pressure of the injection into a painful structure and the inflammatory response these cells cause in the first few days, but within weeks she started to improve. When the MRI was repeated three months later, not only was the majority of her pain gone, so were those MC1 changes (FIG. 7).

According to this disclosure, many patients with Modic Type 1 changes have been treated. Two objectives may be accomplished with one therapeutic, and that is why an intradiscal injection of an LR-PRP is a solution to treat degenerative disc disease (DDD). As disclosed herein, LR-PRP is a safe, effective intradiscal biologic.

### Precision Cell Delivery

When performing interventional spinal procedures, the therapeutic agent should to be delivered as close to the problem as possible to create the greatest possible benefit. Even with an epidural steroid injection, the best effects are when it is placed precisely between the inflamed disc and nerve root.

The problem when an intradiscal injection is performed is that a straight needle is placed into a round structure. Most of the painful tears are in the periphery of the disc. Practitioners are often unable to get a straight needle into that area consistently. Most of the time the needle is placed in the middle of the disc, the injection is performed, and it is hoped that the LR-PRP flows into these tears. It is preferable to have a reliable means of precisely placing the cells as closely into the tears as possible; therefore, with an industry partner, the first intradiscal curved catheter was developed (FIGS. 8A-8C).

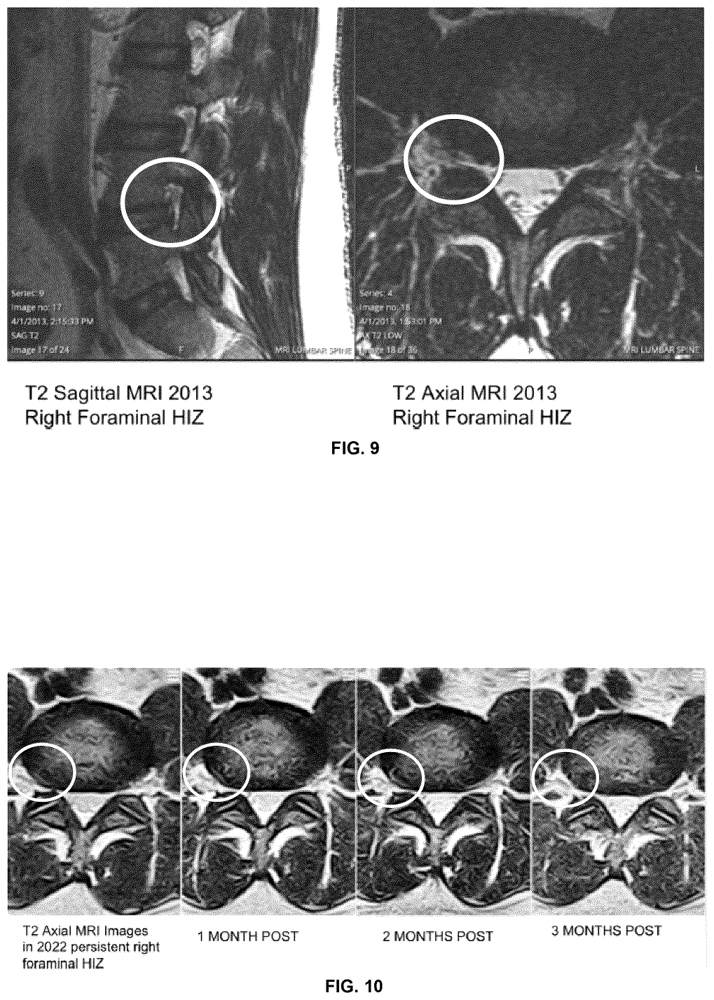

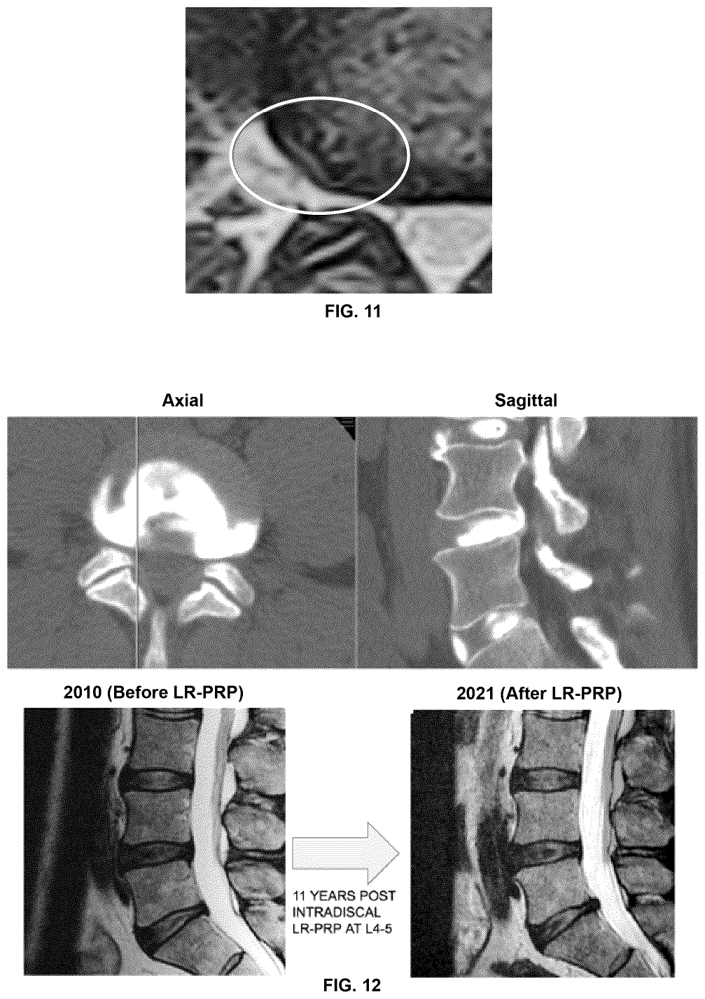

The first in-human test included a 62-year-old gentleman with an 8-year history of low back pain in his right leg who failed conservative treatments. His MRI from 2013 revealed a right foraminal high-intensity zone (HIZ) at L4-5 (the intervertebral disc between lumbar vertebrae 4 and 5), which was unchanged in his most recent MRI from 2022 (FIG. 9). Three months post-treatment, not only were his symptoms completely resolved, but there were documented structural changes in his MRI that showed the resolution of the HIZ (FIG. 10). What was so interesting about this case were the almost immediate annular changes that occurred within 4 weeks of the intra-discal injection (FIG. 11).

### A Promising Future

Chronic back pain is usually an unhealed wound that can be colonized with a certain types of bacteria that can further contribute to degeneration of the disc. Intradiscal leukocyte-rich platelet-rich plasma (LR-PRP), as disclosed herein, is one of the first treatment options that actually targets directly the root causes of chronic low back pain (CLBP) for many. Treatments that just relieve pain and do not create healing structural changes are only palliative, not curative. This is a complete paradigm shift in how CLBP patients are managed in clinical practice.

There are many different types of autologous orthobiologics clinicians are injecting into the disc. There is bone marrow aspirate, bone marrow concentrate, leukocyte poor PRP, platelet lysate, etc. Based on over a decade of clinical experience performing these procedures, the infection risk is the least with LR-PRP. LR-PRP is also the only orthobiologic option that has DB RCT support and long-term outcomes data. Intradiscal bone marrow concentrate has been used in the past, but there are concerns regarding an increased rate of spondylodiscitis for reasons not fully understood (Muthu et al. 2021).

For intradiscal LR-PRP to become the standard of care for patients, multi-center studies would be beneficial to demonstrate its efficacy for specific subsets of patients with degenerative disc disease (DDD) (Prysak et al. 2021). A quality control system that can provide a range of consistency with the LR-PRP preparation method would also be beneficial. PRP can be quantified quickly onsite when compared to other types of cell therapies. With the use of a hemocytometer, one can calculate the delivered dose of platelets, white blood cells, etc. by taking small aliquots of the baseline peripheral blood and the PRP (Prysak et al. 2021). This is relevant to also establish a dosing range that can ultimately be linked to clinical outcomes for optimization. What gets measured gets managed.

The power of regenerative medicine is real, and the field is in the early stages of a paradigm shift in how this global condition can be better managed. It is going to take time, but this shift is already beginning to happen. The regenerative treatments disclosed herein may halt the progression of degeneration. These treatments may not only to provide substantial pain relief, but may also halt or even reverse disc degeneration (FIG. 12).

However, for this paradigm shift to really gain momentum, it is going to be so important for physicians to collaborate not only with the patient, but also with each other, with researchers, with the FDA, hospitals, insurance payors, politicians, and industry to better improve the safety and clinical outcomes of these regenerative procedures.

It would be beneficial for these procedures to be democratized so that anyone suffering from CLBP can have this treatment option before crossing the Rubicon into the surgical maze. It would also be beneficial for the reimbursement challenges to be overcome and payors provided with convincing data on the potential benefit of intradiscal LR-PRP over the current standards of care.

The healthcare system has over-complicated the CLBP problem and mismanaged patients for decades. It has placed patients at unnecessary risk with poorly conceived, ineffectual treatments that have wasted precious healthcare resources. Drugs and surgery have a very limited role in the management of CLBP. It has been rare in 30 years of practice to see a patient totally cured from these types of therapeutics; however, the opposite is now true with the patients treated with intradiscal LR-PRP. Many of these patients have had no further treatments, and their MRIs have demonstrated healing of their degenerated discs for years. Further studies could analyze structural changes in the MRIs of patients treated with intradiscal LR-PRP.

Regenerative medicine is likely to change how patients with CLBP are managed in the years to come. Millions of lives may be improved and billions of healthcare dollars saved when an outpatient injection of a patient's own cells replaces many of those costly hospital-based spinal surgeries. Spinal surgery may still be needed for some patients with more severe later-stage degenerative disc disease that has associated spinal deformity, but nowhere near how many are currently being performed. Intradiscal LR-PRP kills two birds with one stone, finally offering patients an opioid-sparing treatment option that is readily available and scalable. This is value-based musculoskeletal care.

Low back pain (LBP) should be nothing more than a hiccup in life, rather than the wrecking ball it is for so many. Unfortunately, traditional treatments have failed to “fix” low back pain, mainly because only recently has the field really begun to understand the underlying mechanisms that stop a disc from healing. Understanding of why LBP becomes chronic has been rudimentary at best. It is exciting to finally identify new and potentially reversible factors contributing to degenerative disc disease that we can target with regenerative medicine according to the embodiments disclosed herein. If there is a cure for LBP, it lies within this realm of regenerative medicine.

The Regenerative Medicine Revolution is already underway, and it is better for patients if all collaborate to make these treatments as simple, safe, and effective as possible. In the time of personalized and precision medicine, there is none more personalized and precise than the use of a patient's own cells to heal their disc.

When the risk/reward comparison with historical surgical treatments is considered, there really is no comparison. The treatments disclosed herein, in the skilled hands of an interventional spine specialist, are extremely safe and effective. While there are numerous promising regenerative strategies in development, LR-PRP is FDA-compliant and available for patients now. As molecular therapies, biomaterials-based tissue engineering, and other cell therapies evolve, these novel therapies may eventually demonstrate better results than what is already shown herein with LR-PRP, and thereby gain adoption by key opinion leaders.

Finally, the disc should to be thought of as the “heart” of the spine. It should not be cut out or fused, it should be healed and preserved. Maybe if it were treated as such, there would be a chance to preserve the spine and end back pain for many. The field is on the cusp of an exciting new era in regenerative medicine that will change the way LBP is managed so that it does not have to become chronic. Intradiscal LR-PRP is a promising regenerative treatment choice to start this paradigm shift. It is currently a safe orthobiologic intradiscal choice with the most supportive pre-clinical and clinical data.

### EXAMPLE 2

A disclosure similar to the following example is published as “Clinical outcomes following intradiscal injections of higher-concentration platelet-rich plasma in patients with chronic lumbar discogenic pain” in International Orthopaedics (Lutz et al. 2022), the contents of which are herein incorporated in their entirety by reference).

Improved understanding of the pathophysiological basis of chronic lumbar discogenic pain has led to the ongoing development of targeted intradiscal biologic therapies that aim to facilitate healing by delivering autologous growth factors directly to the site of injury. In particular, platelet-rich plasma (PRP) can be beneficial in regions in which vascularity is minimal, such as the intervertebral disc (IVD), due to various factors that are emitted from the platelets and cells. Several studies have shown short term or long-term improvements in patient-reported pain and function following intradiscal injections of PRP.

### Introduction

Low back pain (LBP) is the leading cause of disability worldwide (James et al. 2018), affecting an estimated 577 million people at any time (Wu et al. 2020). The global burden of LBP is considerable. In the United States of America (U.S.A.) alone, the economic burden of LBP is estimated to exceed $250 billion annually in lost wages and treatment costs (United States Bone and Joint Initiative 2014).

The pathophysiology of LBP-while multifactorial-is often associated with damage to the intervertebral disc (IVD). Up to 42% of chronic LBP cases are discogenic in nature (Schwarzer et al. 1995, DePalma et al. 2011, Verrills et al. 2015). The injured IVD has limited self-healing capacity due to its poor vascular supply, hypoxic microenvironment, and low cell content. Lack of access to blood-based growth factors, along with inflammation of the richly innervated disc, is theorized to play a role in the prolonged pain that is experienced by many patients with chronic lumbar discogenic pain (CLDP) (Peng 2013).

Improved understanding of the pathophysiological basis of CLDP has led to the ongoing development of targeted intradiscal biologic therapies that aim to facilitate healing by delivering autologous growth factors directly to the site of injury. In particular, platelet-rich plasma (PRP) can be beneficial in regions in which vascularity is minimal, such as the IVD (Tuakli-Wosornu et al. 2016, Monfett et al. 2016), due to various factors that are emitted from the platelets and cells (Marx & Harrell 2014). Several studies have shown short-term or long-term improvements in patient-reported pain and function following intradiscal injections of PRP (Tuakli-Wosornu et al. 2016, Jain et al. 2020, Akeda et al. 2017, Levi et al. 2016, Cheng et al. 2019).

Although data supporting the use of intradiscal PRP therapy for a subset of patients with CLDP is promising, certain questions remain. Determining the optimal PRP preparation is one such area of clinical interest. Jain et al. demonstrated a positive correlation between higher platelet concentrations and improvements in outcomes in patients receiving intradiscal PRP therapy for discogenic LBP (Jain et al. 2020). The majority of studies in the literature have utilized PRP preparations with a three- to fivefold increase in platelet concentrations (Tuakli-Wosornu et al. 2016, Levi et al. 2016). Newer preparations have yielded higher concentrations of platelets in PRP. The cellular content of PRP that was processed from 118 patients using an EmCyte PURE PRP® II kit (EmCyte Corp., Fort Myers, Fla.) for preparing specially formulated platelet-rich plasma was previously analyzed. Greater than tenfold (>10×) increases in platelet concentration were observed in the PRP that was injected in the majority of patients (Prysak et al. 2021). No study has evaluated patient-reported outcomes following intradiscal injections of PRP with higher platelet concentrations.

The objectives of this study were to (1) determine whether intradiscal injections of PRP with higher platelet concentrations would improve clinical outcomes in patients with CLDP, and (2) compare clinical outcomes to historical data from a previous study utilizing intradiscal injections of PRP with lower platelet concentrations. As disclosed herein, it was hypothesized that intradiscal injections of PRP with higher platelet concentrations would significantly improve pain and functional outcomes in patients with CLDP, and that these improvements would be greater than those observed in a historical cohort of patients receiving intradiscal injections of PRP with lower platelet concentrations.

### Materials and Methods

### Ethics and Patient Selection

This retrospective cohort study was approved by the Institutional Review Board. Written informed consent was waived, as all data were retrospectively collected from patients' charts. Patients with chronic lumbar discogenic pain (CLDP) who received intradiscal platelet-rich plasma (PRP) injections between January 2017 and January 2019 were retrospectively assessed for eligibility at a single outpatient interventional orthopedic clinic. The inclusion criteria were: (1) chronic refractory low back pain greater than leg pain for more than three months; (2) failure of conservative treatments; (3) maintained disc height of at least 50%; (4) lumbar protrusion <4 mm; (5) higher concentration (>10×) intra-discal PRP procedure; (6) absence of any contraindications (e.g., progressive neurologic deficits, severe spinal stenosis); and (7) available post-injection outcome data. The exclusion criteria were: (1) previous spinal fusion surgery; (2) spondylolysis; (3) disc extrusion or sequestered fragment; (4) severe disc degeneration; (5) missing contact information preventing follow-up; and (6) intradiscal PRP combined with another orthobiologic agent. Patients who had degenerative grade I spondylolisthesis or a previous history of microdiscectomy were permitted to be included in the study.

### Procedure Details

PRP was prepared as previously described (Prysak et al. 2021). Briefly, for each level (i.e., disc) injected, 60 ml peripheral blood was drawn and processed down to 4 ml PRP. This yielded platelet counts that were increased by at least 10×, as previously shown (Prysak et al. 2021). A small 3-ml syringe was used to slowly inject the PRP (2 ml per disc) at low pressures over one to two minutes. Patients received either leukocyte-rich PRP or leukocyte-poor PRP.

### Data Collection

Demographic information, duration of pain, and patient-reported outcomes were collected. Baseline patient-reported outcomes included the numerical rating scale (NRS) pain score and Functional Rating Index (FRI). Follow-up patient-reported outcomes included the NRS pain score, FRI, and the North American Spine Society (NASS) Patient Satisfaction Index. The minimum clinically important differences (MCIDs) for NRS pain and the FRI are 2 points and 9 points, respectively (Childs et al. 2005, Copay et al. 2008). Patient satisfaction was defined as a response of “The procedure met my expectations” or “I improved less than I hoped, but I would undergo the same procedure again for the same results.” Follow-up time points ranged from three to 43 months.

### Statistical Analysis

For the descriptive analysis, continuous data are reported as means, standard deviations, and/or ranges, and discrete data are reported as counts and percentages. Patient-reported outcomes data from the 37 patients in the current study were subsequently compared with one-year historical outcomes data from 29 patients from a 2016 double-blind randomized controlled trial (Tuakli-Wosornu et al. 2016), where patients received intradiscal PRP therapy using a lower concentration of platelets (>5×PRP). Independent sample t-tests were used to assess differences in continuous variables, and chi-square tests were used to evaluate differences in discrete variables. A p-value <0.05 was considered to be statistically significant.

### Results

### Patient Flow

Eighty-one patients were retrospectively assessed for eligibility at a single outpatient interventional orthopedic clinic. Forty-four patients did not meet inclusion criteria. The remaining 37 patients (>10×PRP 2021 cohort) were included in the study.

### Demographics and Follow-Up Intervals

The mean age was 42.7±18.2 years (range: 14-72); 12 patients were between 14 and 29 years of age, 15 patients were between 30 and 55 years, and ten patients were between 50 and 72 years. There were 23 males and 14 females. The mean follow-up time from the date of procedure was 18.3±13.3 months (range: 3-43 months; 3-6 months: n=8; 7-12 months: n=7; 13-24 months: n=8; 25-43 months: n=14). All but three patients reported a pain duration for seven months or more prior to the procedure, and 15 patients reported a pain duration for greater than 24 months.

### Clinical Outcomes and Patient Satisfaction

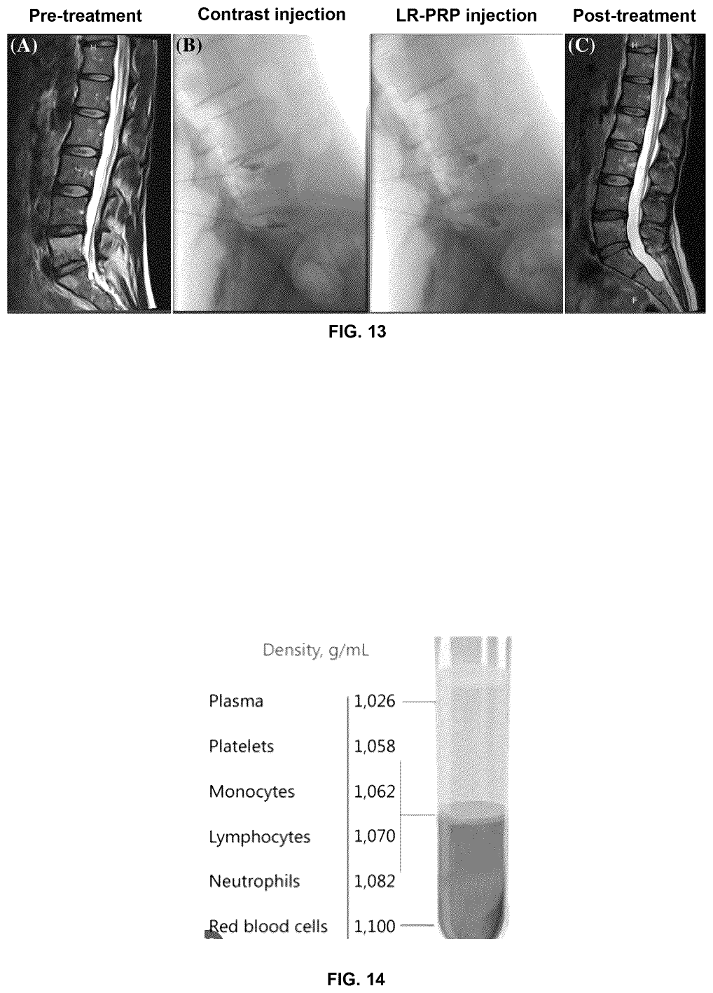

Pain and Functional Rating Index (FRI) scores are reported in Table 2. There were significant improvements in pain and FRI scores at the time of follow-up, with mean changes of 3.4±2.5 and 46.4±27.6% from baseline, respectively (p<0.001 for both). Minimum clinically important differences (MCIDs) in pain and function were met by 73% of patients (n=27) and 89% of patients (n=33), respectively. Representative magnetic resonance imaging scans from a patient who demonstrated improvement following treatment are shown in FIG. 13.

TABLE 2
Pain and functional rating index scores.

| | | | <5 × PRP 2016 | | |
|---|---|---|---|---|---|
| | >10 × PRP 2021 | | (Historical) | | |
| | Cohort (n = 37) | | Cohort (n = 29) | | P-value |
| | | P-value | | P-value | (2021 |
| | Mean | (vs. | Mean | (vs. | vs. |
| | (SD) | baseline) | (SD) | baseline) | 2016) |
| Baseline NRS Pain Score | 6.8 (2.0) | | 4.7 (2.2) | | <0.001 |
| Follow-up NRS Pain Score | 3.4 (2.4) | <0.001 | 3.2 (2.4) | 0.063 | 0.738 |
| Change in NRS Pain from | 3.4 (2.5) | | 1.7 (1.6) | | 0.004 |
| Baseline | | | | | |
| Baseline FRI Score | 65.3 (18.7) | | 51.5 (15.6) | | 0.002 |
| Follow-up FRI Score | 34.3 (21.8) | <0.001 | 33.9 (20.4) | 0.001 | 0.940 |
| Change in FRI Score from | 46.4 (27.6) | | 33.7 (12.3) | | 0.016 |
| Baseline | | | | | |

FRI, Functional Rating Index;
NRS, numerical rating scale;
PRP, platelet-rich plasma;
SD, standard deviation

The majority of patients reported that they started noticing improvements in the first 12 weeks (n=26; 70.3%) and were satisfied with their PRP injection (n=30; 81%). Five (13.5%) patients noted that they were the same or worse than before the procedure and did not experience any improvements. The procedure was defined as successful if patients reported all of the following: (1)≥2-point change in NRS pain score; (2)≥9-point change in the FRI; and (3) satisfaction with the procedure. Twenty-six patients met the criteria for a success rate of 70%. Seven patients (19%) did not meet any of the criteria for success and failed to improve at all. One patient had a complication of spondylodiscitis (Beatty et al. 2019). No patients experienced a progressive disc herniation post-procedure.

### Comparisons to Outcomes in the Historical Cohort

For the historical (<5×PRP 2016) cohort, the mean change in pain score from baseline was 1.7±1.6 (p=0.063), and the mean change in FRI score from baseline was 33.7±12.3 (p=0.001). Compared to the historical cohort, the >10×PRP 2021 cohort started with worse pain and function scores and experienced significantly greater degrees of improvement in both pain and function (p=0.004 and 0.016, respectively) (Table 2). In addition, the patient satisfaction rate was higher in the >10×PRP 2021 cohort compared to the historical cohort (81% vs. 56%; x2=4.9; p=0.027). Of note, age and gender distributions were similar between the >10×PRP 2021 cohort and the historical cohort (42.7±18.2 vs. 41.4±8.1 years; 38% vs. 52% females).

### Discussion

Emerging data have suggested that intradiscal platelet-rich plasma (PRP) therapy with less than fivefold increases in platelet concentrations may have therapeutic value for certain patients with chronic lumbar discogenic pain (CLDP) (Tuakli-Wosornu et al. 2016, Levi et al. 2016, Chang et al. 2021, Muthu et al. 2021). The results disclosed herein add to the current literature showing long-term benefits of intradiscal PRP in the management of patients with CLDP (Monfett et al. 2016, Cheng et al. 2019). There were statistically and clinically significant improvements in patient-reported numerical rating scale (NRS) pain and Functional Rating Index (FRI) scores at an average of 18 months following intradiscal injections of PRP with greater than tenfold increases in platelet concentration. Patient satisfaction and success rates were high. No other study has yet investigated clinical out-comes following intradiscal injections of >10×PRP.

Platelets have been shown to contain more than 1500 bioactive proteins, including growth factors, adhesive proteins, chemokines, and angiogenic factors (Boswell et al. 2012). In particular, platelet-derived growth factor (PDGF) and transforming growth factor β (TGF-β) are abundant in platelets and also play a role in healing (Anitua et al. 2004). Previous studies have demonstrated a positive linear relationship between platelet count and TGF-β or PDGF concentrations in PRP preparations (Sundman et al. 2011). As a correlation between higher platelet concentrations and improved patient-reported outcomes has been reported (Jain et al. 2020), according to this disclosure it was hypothesized that PRP preparations containing higher platelet concentrations (>10×) would further improve pain and functional outcomes in patients with chronic low back pain (CLBP) compared to PRP preparations containing lower platelet concentrations (<5×). This was supported by the findings disclosed herein, which showed significantly greater degrees of improvement in pain and function in patients receiving intradiscal injections of >10×PRP than those receiving <5×PRP from a historical cohort (Tuakli-Wosornu et al. 2016). This was observed despite the current >10×PRP 2021 cohort having worse pain and function at baseline compared to the historical cohort. In addition, patient satisfaction was higher in the current cohort compared to the historical cohort (81% vs. 55%).

The success rate following intradiscal injections of >10 x PRP was high in this cohort of CLDP patients; however, seven patients (19%) failed to improve. Future studies involving further characterization of the PRP injectate, including cell differentials and bioactive protein levels and assessment of magnetic resonance imaging (MRI) criteria, would better elucidate why some patients do not experience any improvements following these injections.

There are several conditions to consider for this study. First, the follow-up time points were variable due to the retrospective nature of the study. Second, baseline NRS pain and FRI scores in the current >10×PRP cohort were worse than those in the historical <5× PRP cohort; however, age and gender distributions were similar between cohorts. Lastly, shorter-term effects were not assessed in the study and should be investigated in the future.

Overall, there is a need for evidence-based, interventional treatments that address the underlying pathology of CLDP. Intradiscal PRP therapy has shown promising results in the CLDP literature, but there are wide variabilities in PRP type and processing. Results from this study suggest that clinical outcomes following intradiscal PRP injections in patients with CLDP can be optimized by using a PRP preparation with a higher concentration of platelets (>10×). Future studies exploring PRP with higher concentrations of platelets in larger sample sizes and with longer follow-ups are warranted, as well as a more detailed analysis of the cellular composition of PRP (e.g., leukocyte-rich vs. leukocyte-poor) to assess other variables.

EXAMPLE 3

In some aspects, methods of the disclosure may be carried out using a delivery device for delivering a therapeutic agent to the disc of the spine as described in WO 2020/160444, the contents of which are herein incorporated in their entirety by reference. For example, such a delivery device can include an introducer needle having a hollow cannula, a stylet, and a delivery catheter with a hollow shaft. The catheter is configured to fit coaxially through the introducer needle when the stylet is removed. The catheter is longer than the introducer needle and has a blunt closed-ended tip and at least one side port at the distal end. The introducer needle and stylet have a sharpness and consistency that allows them to penetrate the wall of the annulus fibrosus. Advantageously, the catheter forms a pre-set bend as it exits coaxially through the distal end of the introducer needle. The angle of the bend of the catheter in combination with the blunt tip allow the catheter to traverse inside the circumference of the annulus fibrosus without entering the nucleus pulposus or exiting the annulus fibrosus into surrounding tissue. A biologic (e.g., autologous platelet rich plasma, bone marrow aspirate, bone marrow concentrate, fibrin, or non-autologous fibrin or cells, or a combination thereof), including PRP compositions described herein, can be injected through the delivery catheter.

### REFERENCES

Akeda K, An H S, Pichika R, et al. (2006) Platelet-rich plasma (PRP) stimulates the extra-cellular matrix metabolism of porcine nucleus pulposus and annulus fibrosus cells cultured in alginate beads. Spine (Phila Pa. 1976). 2006;31(9):959-66.

Akeda K, Ohishi K, Masuda K, Bac W C, Takegami N, Yamada J, Nakamura T, Sakakibara T, Kasai Y, Sudo A (2017) Intradiscal injection of autologous platelet-rich plasma releasate to treat discogenic low back pain: a preliminary clinical trial. Asian Spine J 11:380-389. https://doi.org/10.4184/asj.2017.11.3.380

Akeda K, Yamada J, Linn E T, et al. (2019) Platelet-Rich Plasma in the Management of Chronic Low Back Pain: A Critical Review. J Pain Res 2019; 12:753-67.

Akeda K, Takegami N, Yamada J, et al. (2022a) Platelet-Rich Plasma-Releasate (PRPr) for the Treatment of Discogenic Low Back Pain Patients: Long-Term Follow-Up Survey. Medicina (Kaunas) 2022;58(3):428. PMCID: PMC8952290.

Akeda K, Ohishi K, Takegami N, et al. (2022b) Platelet-Rich Plasma Releasate versus Corticosteroid for the Treatment of Discogenic Low Back Pain: A Double-Blind Randomized Controlled Trial. J Clin Med 2022; 11(2):304. PMID: 35053999; PMCID: PMC8777786.

Albert H B, Sorensen J S, Christensen B S, et al. (2013) Antibiotic treatment in patients with chronic low back pain and vertebral bone edema (Modic type 1 changes): a double-blind randomized clinical controlled trial of efficacy. Eur Spine J 2013; 22(4):697-707. Epub 2013 Feb 13. PMID: 23404353; PMCID: PMC3631045.

Anitua E, Andia I, Ardanza B, Nurden P, Nurden A T (2004) Autologous platelets as a source of proteins for healing and tissue regeneration. Thromb Haemost 91:4-15. https://doi.org/10.1160/th03-07-0440

Beatty N R, Lutz C, Boachie-Adjei K, Leynes T A, Lutz C, Lutz G (2019) Spondylodiscitis due to Cutibacterium acnes following lumbosacral intradiscal biologic therapy: a case report. Regen Med 14:823-829. https://doi.org/10.2217/rme-2019-0008

Boswell S G, Cole B J, Sundman E A, Karas V, Fortier L A (2012) Platelet-rich plasma: a milieu of bioactive factors. Arthroscopy 28:429-439. https://doi.org/10.1016/j.arthro.2011.10.018

Centers for Disease Control and Prevention (2018). 2018 Annual Surveillance Report of Drug-related Risks and Outcomes—United States. Washington, D.C.: U.S. Department of Health and Human Services; 2018.

Chang M C, Park D (2021) The effect of intradiscal platelet-rich plasma injection for management of discogenic lower back pain: a meta-analysis. J Pain Res 14:505-512. https://doi.org/10.2147/jpr.S2923 35

Chen W H, Lo W C, Lee J J, et al. (2006) Tissue-engineered intervertebral disc and chondrogenesis using human nucleus pulposus regulated through TGF-betal in platelet-rich plasma. J Cell Physiol 2006;209(3):744-54.

Chen Z, Zheng Y, Yuan Y, et al. (2016) Modic Changes and Disc Degeneration Caused by Inoculation of Propionibacterium acnes inside Intervertebral Discs of Rabbits: A Pilot Study. Biomed Res Int 2016;2016:9612437.

Cheng J, Santiago K A, Nguyen J T, Solomon J L, Lutz G E (2019) Treatment of symptomatic degenerative intervertebral discs with autologous platelet-rich plasma: follow-up at 5-9 years. Regen Med 14:831-840. https://doi.org/10.2217/rme-2019-0040

Childs J D, Piva S R, Fritz J M (2005) Responsiveness of the numeric pain rating scale in patients with low back pain. Spine (Phila Pa. 1976) 30:1331-1334. https://doi.org/10.1097/01.brs 0000164099.92112.29

Copay A G, Glassman S D, Subach B R, Berven S, Schuler T C, Carreon L Y (2008) Minimum clinically important difference in lumbar spine surgery patients: a choice of methods using the Oswestry Disability Index, Medical Outcomes Study questionnaire Short Form 36, and pain scales. Spine J 8:968-974. https://doi.org/10.1016/j.spinec.2007.11.006

Deng H, Yue J K, Ordaz A, et al. (2011) Elective lumbar fusion in the United States: national trends in inpatient complications and cost from 2002-2014. J Neurosurg Sci 2021;65(5):503-12.

DePalma M J, Ketchum J M, Saullo T (2011) What is the source of chronic low back pain and does age play a role? Pain Med 12:224-233. https://doi.org/10.1111/j.1526-4637.2010.01045.x

Deyo R A, Von Korff M, Duhrkoop D. (2015) Opioids for low back pain. BMJ 2015;350: g6380. PMID: 25561513; PMCID: PMC6882374.

Dieleman J L, Cao J, Chapin A, et al. (2020) US health care spending by payer and health condition, 1996-2016. JAMA 2020;323:863-84.

Donelson R, McIntosh G, Hall H. (2012) Is it time to rethink the typical course of low back pain? PM&R 2012;4(6):394-401. Epub 2012 Mar 3. PMID: 22381638.

Gilligan C J, Cohen S P, Fischetti V A, et al. (2021) Chronic low back pain, bacterial infection and treatment with antibiotics. Spine J 2021;21(6):903-14. Epub 2021 Feb 19. PMID: 33610802.

Grunhagen T, Wilde G, Soukane D M, et al. (2006) Nutrient supply and intervertebral disc metabolism. J Bone Joint Surg Am 2006;88(Suppl 2):30-5. PMID: 16595440.

IOM (Institute of Medicine) (2011). Relieving pain in America: a blueprint for transforming prevention, care, education, and research. Washington, D.C., USA: Institute of Medicine; 2011. p. 5.

Jain D, Goyal T, Verma N, Paswan A K, Dubey R K (2020) Intradiscal platelet-rich plasma injection for discogenic low back pain and correlation with platelet concentration: a prospective clinical trial. Pain Med 21:2719-2725. https://doi.org/10.1093/pm/pnaa254

James, Spencer L. et al. (2018). Global, regional, and national incidence, prevalence, and years lived with disability for 354 diseases and injuries for 195 countries and territories, 1990-2017: a systematic analysis for the Global Burden of Disease Study 2017. Lancet 2018; 392(10159): 1789-858.

Jerome M A, Lutz C, Lutz G E. (2021) Risks of Intradiscal Orthobiologic Injections: A Review of the Literature and Case Series Presentation. Int J Spine Surg 2021; 15(s1): 26-39. Apr 21. PMID: 34376494; PMCID: PMC8092939.

Kho Z Y, Lal S K. (2018) The human gut microbiome-a potential controller of wellness and disease. Front Microbiol 2018. https://doi.org/10.3389/fmicb.2018.01835.

Kirkaldy-Willis W H, Wedge J H, Yong-Hing K, et al. (1976) Pathology and pathogenesis of lumbar spondylosis and stenosis. Spine (Phila Pa. 1976) 1978;3(4):319-28. PMID: 741238.

Levi D, Horn S, Tyszko S, Levin J, Hecht-Leavitt C, Walko E (2016) Intradiscal platelet-rich plasma injection for chronic discogenic low back pain: preliminary results from a prospective trial. Pain Med 17:1010-1022. https://doi.org/10.1093/pm/pnv053

Lo Y T, Lim-Watson M, Seo Y, et al. (2020) Long-Term Opioid Prescriptions After Spine Surgery: A Meta-Analysis of Prevalence and Risk Factors. World Neurosurg 2020; 141:e894-920.

Lutz G E (2022) Intradiscal Leukocyte Rich Platelet Rich Plasma for Degenerative Disc Disease. Phys Med Rehabil Clin N Am. 2023 Feb;34(1):117-133. doi: 10.1016/j.pmr.2022.08.009. Epub 2022 Oct 18. PMID: 36410879.

Lutz C, Cheng J, Prysak M, et al. (2022) Clinical outcomes following intradiscal injections of higher-concentration platelet-rich plasma in patients with chronic lumbar discogenic pain. Int Orthop 2022. https://doi.org/10.1007/s00264-022-05389-y. Epub ahead of print. PMID: 35344055.

Marx R E, Harrell D B (2014) Translational research: the CD34+ cell is crucial for large-volume bone regeneration from the milieu of bone marrow progenitor cells in craniomandibular reconstruction. Int J Oral Maxillofac Implants 29:e201-209. https://doi.org/10.11607/jomi.te56

Monfett M, Harrison J, Boachie-Adjei K, Lutz G (2016) Intradiscal platelet-rich plasma (PRP) injections for discogenic low back pain: an update. Int Orthop 40:1321-1328. https://doi.org/1007/s00264-016-3178-3

Muthu S, Jeyaraman M, Chellamuthu G, Jeyaraman N, Jain R, Khanna M (2021) Does the intradiscal injection of platelet rich plasma have any beneficial role in the management of lumbar disc disease? Global Spine J:2192568221998367. https://doi org/10.1177/2192568221998367

Peng B-G (2013) Pathophysiology, diagnosis, and treatment of discogenic low back pain. World J Orthop 4:42-52. https://doi.org/10.5312/wjo.v4.i2.42

Prysak M H, Lutz C G, Zukofsky T A, et al. (2019) Optimizing the safety of intradiscal platelet-rich plasma: an in vitro study with Cutibacterium acnes. Regen Med 2019;14(10):955-67. Epub 2019 Oct 7. PMID: 31587600.

Prysak M H, Kyriakides C P, Zukofsky T A, Reutter S E, Cheng J, Lutz G E (2021) A retrospective analysis of a commercially available platelet-rich plasma kit during clinical use. PM&R. https://doi.org/10.1002/pmrj.12569

Scholl L, Seth P, Kariisa M, et al. (2018) Drug and Opioid-Involved Overdose Deaths—United States, 2013-2017, MMWR Morb Mortal Wkly Rep, 67(5152);2018:1419-1427.

Schwarzer A C, Aprill C N, Derby R, Fortin J, Kine G, Bogduk N (1995) The prevalence and clinical features of internal disc disruption in patients with chronic low back pain. Spine (Phila Pa. 1976) 20:1878-1883. https://doi.org/10.1097/00007632-19950 9000-00007

Sundman E A, Cole B J, Fortier L A (2011) Growth factor and catabolic cytokine concentrations are influenced by the cellular composition of platelet-rich plasma. Am J Sports Med 39:2135-2140. https://doi.org/10.1177/0363546511417792

Tuakli-Wosornu Y A, Terry A, Boachie-Adjei K, et al. Lumbar Intradiskal Platelet-Rich Plasma (PRP) Injections: A Prospective, Double-Blind, Randomized Controlled Study. PM&R 2016;8(1): 1-10. Epub 2015 Aug 24. PMID: 26314234. https://doi.org/10.1016/j.pmrj.2015.08.010

United States Bone and Joint Initiative (2014) The Burden of Musculoskeletal Diseases in the United States (BMUS), Third Edition. Rosemont, Ill.

Verrills P, Nowesenitz G, Barnard A (2015) Prevalence and characteristics of discogenic pain in tertiary practice: 223 consecutive cases utilizing lumbar discography. Pain Med 16:1490-1499. https://doi.org/10.1111/ pme.12809

Vos, Theo et al. (GBD 2019 Diseases and Injuries Collaborators). Global burden of 369 diseases and injuries in 204 countries and territories, 1990-2019: a systematic analysis for the Global Burden of Disease Study 2019. Lancet (London, England) 2020; 396(10258): 1204-22.

Wu A, March L, Zheng X, Huang J, Wang X, Zhao J, Blyth F M, Smith E, Buchbinder R, Hoy D (2020) Global low back pain prevalence and years lived with disability from 1990 to 2017: estimates from the Global Burden of Disease Study 2017. Ann Transl Med 8:299. https://doi.org/10.21037/atm. 2020.02.175

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of treating, preventing, or ameliorating a tissue disease in a subject, comprising administering to the subject a therapeutically effective amount of a platelet-rich plasma (PRP) composition comprising:
a) plasma;
b) platelets at a concentration of at least 5× to at least 75× the concentration of platelets in whole blood; and
c) nucleated cells at a concentration of at least 5× to at least 75× the concentration of nucleated cells in whole blood, wherein the nucleated cells comprise monocytes, lymphocytes, granulocytes, or a combination thereof.

2. The method of claim 1, wherein the concentration of the platelets, the nucleated cells, the monocytes, the lymphocytes, the granulocytes, or a combination thereof is each at least 10× the concentration in whole blood.

3. The method of claim 1, wherein the treating, preventing, or ameliorating the tissue disease further comprises regenerating tissue.

4. The method of claim 1, wherein the treating, preventing, or ameliorating the tissue disease further comprises reducing bacterial dysbiosis.

5. The method of claim 4, wherein the bacterial dysbiosis comprises an overgrowth of Pseudomonas, Streptococcus, Cutibacterium, or a combination thereof.

6. The method of claim 1, wherein the tissue is avascular tissue.

7. The method of claim 1, wherein the tissue is an intervertebral disc.

8. The method of claim 7, wherein the PRP is delivered intradiscally through an injection.

9. The method of claim 1, wherein the PRP is derived from whole blood.

10. The method of claim 1, wherein the PRP is derived from the subject's own whole blood.

11. The method of claim 1, wherein the PRP is administered in combination with at least one other therapeutic.

12. The method of claim 1, wherein the PRP is administered in combination with an antibiotic.

13. A platelet-rich plasma (PRP) composition comprising:
a) plasma;
b) platelets at a concentration of at least 5× to at least 75× the concentration of platelets in whole blood; and
c) nucleated cells at a concentration of at least 5× to at least 75× the concentration of nucleated cells in whole blood, wherein the nucleated cells comprise monocytes, lymphocytes, granulocytes, or a combination thereof.

14. The composition of claim 13, wherein the concentration of the platelets, the nucleated cells, the monocytes, the lymphocytes, the granulocytes, or a combination thereof is each at least 10× the concentration in whole blood.

15. The composition of claim 13 for use in treating, preventing, or ameliorating a tissue disease; regenerating tissue; reducing bacterial dysbiosis in a tissue; or a combination thereof.

16. The composition of claim 13, wherein the tissue is avascular tissue.

17. The composition of claim 13, wherein the tissue is intervertebral disc tissue.

18. A method of making the composition of claim 13, comprising:
a) centrifuging blood in a first container, thereby producing an upper fraction comprising plasma, platelets, and nucleated cells;
b) transferring the first upper fraction from the first container into a second container;
c) centrifuging the first upper fraction in the second container, thereby producing a second upper fraction comprising plasma; and
d) reducing the volume of plasma in the second upper fraction.

19. The method of claim 18, wherein the first upper fraction is substantially free of red blood cells.

20. The method of claim 18, wherein the step of transferring the first upper fraction comprises manual decanting.
